(19) United States
(12) Patent Application Publication
(10) Pub. No.: US 2012/0226258 A1
(43) Pub. Date: Sep. 6, 2012

(54) DEVICE AND METHOD FOR ELIMINATING BIOLOGICALLY HARMFUL SUBSTANCES FROM BODILY FLUIDS

(76) Inventors: Veit Otto, Berlin (DE); Michaela Hajek, Berlin (DE)

(21) Appl. No.: 13/388,640

(22) PCT Filed: Aug. 9, 2010
(86) PCT No.: PCT/DE10/00954

§ 371 Date: May 22, 2012

(30) Foreign Application Priority Data
Aug. 7, 2009 (DE) 10-2009-0370153

(51) Int. Cl. A61M 1/18

(52) U.S. Cl. 604/500; 422/48

(57) ABSTRACT

A device for purification of blood, blood substitutes or solutions for the introduction into the human and/or animal blood circulation and for gas exchange in blood, blood substitutes or solutions for the introduction into the human and/or animal blood circulation, includes at least one gas permeable membrane and a carrier, coated with substances for adsorptive removal of toxins of biological and chemical synthetic origin, their metabolites and degradation products present in blood, blood substitutes or solutions for the introduction into the human and/or animal blood circulation, a use of the aforementioned device and a process for gentle and simultaneous removal of toxins of biological and chemical synthetic origin, their metabolites and degradation products present in blood, blood substitutes or solutions for the introduction into the human and/or animal blood circulation and for enrichment of blood, blood substitutes or solutions for the introduction into the human and/or animal blood circulation with oxygen.

### BACKGROUND OF THE INVENTION

1. Field of the Invention

The following invention generally relates to a device for purification of blood, blood substitutes or solutions for the introduction into the human and/or animal blood circulation and for gas exchange in blood, blood substitutes or solutions for the introduction into the human and/or animal blood circulation.

2. Description of the Relevant Art

The removal of harmful substances in blood has been practiced for a long time. Thereby the dialysis procedures which are performed in acute and chronic renal failure have to be mentioned in the first place. The development of these procedures, since the initial application in 1924, led to a globally recognized and successfully practiced life-saving or life-prolonging measure for people with renal failure. In the field of dialysis for the past 20 years in particular the successful use of hollow fiber adsorbers from Fresenius AG can be mentioned.

Since then, these extracorporeal procedures have been used in many areas of medicine, when it comes to free body liquids from harmful substances or to perform an exchange of substances. The apparatus used therefore have been developed usually for a specific task. Such as dialysator purifies the plasma of patients from waste products of the metabolism during hemodialysis as “artificial kidney”, problems of the immune system can also be solved with the help of adsorbers. After separation of blood cells, the blood plasma is passed over an apheresis column where the pathogenic antibodies are selectively bound and the purified blood plasma is then returned to the patient. For these cases, the columns must have the necessary specific binding sites for these antibodies to be able to bind these antibodies.

Although possibilities are increasing to improve life-threatening conditions by the removal of the causes present in the body fluids, until today there is still a great need for haemo-compatible materials as well as gentle and effective working methods of removing toxic substances from body fluids as well as from contaminated solutions for introduction to the body.

At the same time, there is an increasing need for therapies that deal with secondary diseases, because often not the initial disease is lethal but the complications occurring as a result of the initial disease. A prominent example is sepsis, which is currently in 10th place on the list of leading causes of death and whose occurrence is increasing steadily. Since 30% to 50% of the patients suffering from sepsis die, despite maximal therapy, it represents a very serious problem. Additionally, the increasing occurrence of bacteria resistant to antibiotics is already a serious and growing problem in hospitals.

Sepsis is caused by the occurrence of inflammation, which may generally occur after injury and in hospital usually after surgery. Similarly, nosocomial infections still play an important role in daily clinical practice. For example, catheter-associated bloodstream infections are still frequent complications.

The inflammation activated immune system attacks existing gram-negative bacteria (e.g. Escherichia coli, Pseudomonas aeruginosa, Enterobacter aerogenes, Salmonella, Shigella, Neisseria, such as meningococci and the causative agent of gonorrhoea). This is followed by the lysis of bacteria and removal of the degradation products through the bloodstream to the kidneys. These degradation products include cell membrane components, which are distributed as enterotoxins, endotoxins or lipopolysaccharides (LPS) in the whole organism and exert their toxic effect. In those cases where the immune defense of the body is not able to stop the inflammation process, the situation gets out of control and the infection develops into a sepsis.

As a standard therapy against sepsis, the patient usually receives an antibiotic which has been tested beforehand microbiologically for its effectiveness against the bacteria. If an organ dysfunction has already established, this organ must be supported in its function (organ support therapy) or the organ must be replaced temporarily (organ replacement therapy). Respiratory and circulatory systems must be stabilized at this stage. If these measures do not suffice, further organ failure is the result ultimately leading to death due to multiorgan failure. A particularly serious case occurs, when the necessary administration of antibiotics leads to a sharp increase of endotoxins caused by the rapid killing of the bacteria, so that the pathophysiological processes are accelerated immensely, or in another case the bacteria are resistant against the antibiotics and thus no standard therapy is possible anymore.

To date there exist no adsorbents that successfully remove the sepsis-causing endotoxins from the blood. Various approaches with adsorbers did not show the expected positive effect.

For example, DE 19648954A1 describes an endotoxin adsorber working with a particulate carrier, wherein covalent amine or ammonium group-containing ligands are coupled. DE 4113602A1 describes an endotoxin adsorber with pearled cellulose products as a carrier material and polyethylenimine as a ligand, whereas in DE 102006055558A1 the carrier material consists of a polysaccharide in any form and the amino group containing ligand preferably is polyallylamine or polyethylenimine.

A research group in Munich tried to succeed by coupling L-arginine to a carrier. Besides, although beneficial side effects have been achieved, the feasibility study that was conducted on 10 patients showed, that after conduction of plasmapheresis the concentration of endotoxins was still undiminished and so the removal of endotoxins was unsuccessful (Blood Purif. 2008; 26: 333-339).

Thus, there remains a great need for effective solutions for the removal of harmful substances from the blood, blood substitutes or solutions for the introduction into the human and/or animal blood circulation, especially for the removal of endotoxins from whole blood and the effective treatment of sepsis.

### SUMMARY OF THE INVENTION

Object of the present invention is to provide a device and methods which remove effectively toxins of biological and chemical synthetic origin, their metabolites and degradation products from blood, blood substitutes or solutions for the introduction into the human and/or animal blood circulation, especially for the effective treatment of sepsis.

This object is achieved by the technical teaching of the independent claims. Further advantageous embodiments of the invention will become apparent from the dependent claims, the description and examples.

A device for purification of blood, blood substitutes or solutions for the introduction into the human and/or animal blood circulation and for gas exchange in blood, blood substitutes or solutions for the introduction into the human and/or animal blood circulation, comprising at least one gas permeable membrane and a carrier, coated with substances for adsorptive removal of toxins of biological and chemical synthetic origin, their metabolites and degradation products present in blood, blood substitutes or solutions for the introduction into the human and/or animal blood circulation, a use of the aforementioned device and a process for gentle and simultaneous removal of toxins of biological and chemical synthetic origin, their metabolites and degradation products present in blood, blood substitutes or solutions for the introduction into the human and/or animal blood circulation and for enrichment of blood, blood substitutes or solutions for the introduction into the human and/or animal blood circulation with oxygen.

In an embodiment, a device for purification of blood, blood substitutes or solutions for the introduction into the human and/or animal blood circulation and for gas exchange in blood, blood substitutes or solutions for the introduction into the human and/or animal blood circulation comprises a column with:
a) an inlet and an outlet for gases or gas mixtures,
b) an inlet and an outlet for blood, blood substitutes or solutions for the introduction into the human and/or animal blood circulation,
c) at least one gas permeable membrane and
d) a carrier, which is coated with substances for adsorptive removal of toxins of biological and chemical synthetic origin, their metabolites and degradation products present in blood, blood substitutes or solutions for the introduction into the human and/or animal blood circulation.

The device may be used for removal of toxins of biological and chemical synthetic origin, their metabolites and degradation products present in blood, blood substitutes or solutions for the introduction into the human and/or animal blood circulation

### DETAILED DESCRIPTION OF THE PREFERRED EMBODIMENTS

Surprisingly it has been found that the extracorporeal removal of endotoxins from blood by adsorption to an endotoxinaffine substance for the treatment of sepsis and the simultaneously extracorporeal organ replacement or support therapy can be done successfully by using the same device and adsorber column, so that such a combined device, performs two functions simultaneously. This leads in many regards to a significant improvement in therapy. On the one hand the same apparatus removes with a single application the life-threatening endotoxins from the blood of the patient, and on the other hand, the sepsis-induced diseased organ is supported until decreasing concentrations of endotoxins allow the organ to fulfill its function again. This results in an optimum coupling of an active curative effect and of a component supporting the survival function. Moreover, in this system the burden on the patients is far below that of the usual procedures, which also increases the chances of recovery of the fatally ill patient. Another important advantage of using such a dual system are the savings in time. As previously mentioned, the acute life-threatening condition can occur within minutes, so that little time remains for further therapeutic measures. Such situations can be avoided a priori with the use of the double-functional device. Therefore, by a timely treatment with the devices described herein one can avoid an acute sepsis shock and thus save the patient's life.

In a preferred embodiment in sepsis-induced reduction of lung function an extracorporeal membrane oxygenation (ECMO) is performed in which a membrane oxygenator exchanges oxygen and carbon dioxide in blood, wherein the oxygenator membrane is coated with endotoxin-binding substances for the removal of endotoxins in the blood and thus for the elimination of sepsis-causing toxins. Of course, the coated oxygenator membrane can be used as an endotoxine adsorber only. This preferred embodiment can be described as an oxygenator-endotoxine adsorber.

In this way, the use of an extracorporeal organ support apparatus fulfills a dual function. Firstly, the necessary measures are carried out in organ dysfunction and simultaneously without extra effort during extracorporeal oxygenation of the blood, with the same device and the same membrane module also the endotoxins are filtered from the blood and thus a therapeutically important measure to cure the patient is achieved.

Also in another preferred embodiment, hemofiltration as renal replacement therapy can be combined with extracorporeal oxygenation and the removal of endotoxins, resulting in a triple function, wherein the oxygenation membrane and/or the filtration membrane of hemofiltration is coated with endotoxine binding substances. Besides the support of the renal function and the lung function, the device with the triple function is also capable to eliminate endotoxins from the blood.

A device thus fulfills a double function. One of its two functions consists of the purification of blood, blood substitutes or solutions for the introduction into the human and/or animal blood circulation. The second function is the gas exchange, i.e. the enrichment with oxygen and removement of carbon dioxide in blood, blood substitutes or solutions for the introduction into the human and/or animal blood circulation. Both functions are fulfilled by the device simultaneously.

This device with a dual function comprises a column I with an inlet and possibly an outlet for gases or gas mixtures, an inlet and an outlet for blood, blood substitutes or solutions for the introduction into the human and/or animal blood circulation, at least one gas-permeable membrane, and a carrier that is coated with substances for adsorptive removal of toxins of biological and chemical synthetic origin, their metabolites and degradation products in blood, blood substitutes or solutions for the introduction into the human and/or animal blood circulation.

The column I can have in addition to the at least one inlet and the at least one optional outlet for gases or gas mixtures, multiple inlets and/or multiple outlets. Furthermore, the column can include one or more inlets and/or one or more outlets for blood, blood substitutes or solutions for the introduction into the human and/or animal blood circulation circulation.

The term “blood” is to be understood as blood, whole blood, blood plasma and blood serum. The term “blood substitute” is to be understood as blood substitutes that e.g. at least in part can take over actively the oxygen transport, and volume expanders thinning the remaining blood and complement it insofar that the blood circulation works again, but bear no physiological function of the blood itself.

The term “solutions for the introduction into the human and/or animal blood circulation” is to be understood as pharmaceutical preparations and pharmaceutical concentrates for intravenous, intraarterial or intracardiac administration, such as physiological saline solution, artificial nutrition media for artificial nutrition, contrast agents, in particular for imaging techniques such as X-ray contrast media as well as injection solutions comprising pharmaceutical drugs such as antiproliferative or anti-inflammatory or anti-angiogenic or anti-viral or antibacterial or antiparasitic drugs.

The device with dual function consists of a column I, which is divided by a gas permeable membrane into a first chamber and a second chamber. Here, the first chamber is formed by the inner space of the column. The gas-permeable membrane may consist of one or more bundles of hollow fibers. In the event that the gas-permeable membrane is present in the form of one or more bundles of hollow fibers, the second chamber is formed by the inner space of the one or several bundles of hollow fibers, which are arranged in the column. The bundle or the bundles of hollow fibers are arranged so that one of its ends opens at least into one of the inlets and the other end into at least one of the outlets. By this way, blood or blood substitutes or solutions for the introduction into the human and/or animal blood circulation or gas or a gas mixture can flow through the second chamber. The inner space of the column is also connected to at least one inlet and/or at least one outlet, so that blood or blood substitutes or solutions for the introduction into the human and/or animal blood circulation or gas or a gas mixture can also flow through the first chamber. The column has a substantially cylindrical shape, but other functional forms are possible.

Two embodiments are possible. In one embodiment, the first chamber is flown through by blood, blood substitutes or solutions for the introduction into the human and/or animal blood circulation and the second chamber is flown through by gas or a gas mixture. In another embodiment, the second chamber is flown through by blood, blood substitutes or solutions for the introduction into the human and/or animal blood circulation and the first chamber is flown through by gas or a gas mixture.

The carrier, which is coated with substances for adsorptive removal of toxins of biological and chemical synthetic origin, their metabolites and degradation products in blood, blood substitutes or solutions for the introduction into the human and/or animal blood circulation, can take the form of particles or the form of hollow fibers. The carrier, which is coated with substances for adsorptive removal of toxins of biological and chemical synthetic origin, their metabolites and degradation products in blood, blood substitutes or solutions for the introduction into the human and/or animal blood circulation, is hereafter simply referred to as a carrier. If the carrier is present in the form of particles, then the carrier-particles of the two above-mentioned embodiments are located respectively in the chamber, which is flown through by blood, blood substitutes or solutions for the introduction into the human and/or animal blood circulation. The chamber flown through by gas contains no carrier-particles. If the carrier is provided in form of hollow fibers, the carrier and the gas permeable membrane is combined into a single unit or rather form a unit.

In addition, the device may have a third function. The third function consists in the support of renal function by hemofiltration. The device with triple functionality therefore accomplishes the support of renal function, the support of lung function and the removal of toxins of biological and chemical synthetic origin, their metabolites and degradation products in blood, blood substitutes or solutions for the introduction into the human and/or animal blood circulation.

The device with triple functionality comprises the above-described column, in the following referred to as column I, as well as a further column, which is referred to as column II. The column II comprises in turn one or more outlets for filtrate, at least one semi-permeable membrane, and a carrier coated with substances for adsorptive removal of toxins of biological and chemical synthetic origin, their metabolites and degradation products in blood, blood substitutes or solutions for the introduction into the human and/or animal blood circulation. Furthermore, the column II may include one or more inlets and/or one or more outlets for blood, blood substitutes or solutions for the introduction into the human and/or animal blood circulation. In the device with triple functionality, the two columns, column I and column II, are connected in series, which means that the blood, blood substitutes, or the solutions to be introduced into the human and/or animal blood circulation first pass through one column and then the other column. Thereby optionally either column I is flown through first and then column II, or the two columns are flown through in the reverse order. Preferably, the blood, blood substitutes or the solutions to be introduced into the human and/or animal blood circulation flow through column II (haemofiltration and where appropriate the removal of toxins) before column I (oxygen/carbon dioxide exchange and removal of toxins).

Hence, the device with triple functionality can optionally use only column I for the purification of blood, blood substitutes or solutions for the introduction into the human and/or animal blood circulation or rather for removing toxins of biological and chemical synthetic origin, their metabolites and degradation products in blood, blood substitutes or solutions for the introduction into the human and/or animal blood circulation. Or the device with triple functionality can use additionally to column I also column II for this function. Thus, the binding capacity of the device with triple functionality for toxins of biological and chemical-synthetic origin, their metabolites and degradation products is doubled and cleansing effect is increased considerably.

The column II is divided by a semipermeable membrane into a first chamber and a second chamber. Here, the first chamber is formed by the inner space of the column. The semipermeable membrane can consist of one or more bundles of hollow fibers. In the event that the semipermeable membrane is provided in the form of one or more bundles of hollow fibers, the second chamber is formed by the inner space of one or several bundles of hollow fibers, which are arranged in the column. The bundle or the bundles of hollow fibers can be arranged so that one of its ends opens at least into one of the inlets and the other end into at least one of the outlets. In this arrangement the blood, blood substitutes, or the solution for introduction into the human and/or animal blood circulation flows through the second chamber. If the bundle or bundles of hollow fibers is/are arranged so that both ends lead into at least one of the outlets, the second chamber is used for collecting and discharging the filtrate. The inner space of the column II can also be connected to at least one inlet and/or at least one outlet, so that the first chamber is also flown through by blood, blood substitutes or solutions for the introduction into the human and/or animal blood circulation. If the inner space of the column includes at least one outlet, the first chamber is used for collecting and discharging the filtrate. The column II has a substantially cylindrical shape; but other functional forms are possible.

Thereby two embodiments are possible. In one embodiment, the first chamber is flown through by blood, blood substitutes or solutions for the introduction into the human and/or animal blood circulation and the second chamber is used for collecting and discharging the filtrate. In another embodiment, the second chamber is flown through by blood, blood substitutes or solutions for the introduction into the human and/or animal blood circulation and the first chamber is used to collect and discharge of the filtrate.

The carrier, which is coated with substances for adsorptive removal of toxins of biological and chemical synthetic origin, their metabolites and degradation products in blood, blood substitutes or solutions for the introduction into the human and/or animal blood circulation, can take the form of particles or possess the form of hollow fibers. The carrier, which is coated with substances for adsorptive removal of toxins of biological and chemical synthetic origin, their metabolites and degradation products in blood, blood substitutes or solutions for the introduction into the human and/or animal blood circulation, is hereafter simply referred to as a carrier. If the carrier is present in the form of particles, then the carrier-particles of the two above-mentioned embodiments are located respectively in the chamber, which is flown through by blood, blood substitutes or solutions for the introduction into the human and/or animal blood circulation. The chamber used for collecting and discharging the filtrate does not contain any carrier-particles. If the carrier is provided in form of hollow fibers, the carrier and the gas permeable membrane are combined into a single unit or rather form a unit.

Both devices comprise diverse tube connections, a filter unit, a pump and not necessarily but advantageously a tempering unit. The tempering unit ensures that the temperature of blood, blood substitutes or the solutions to be introduced into the human and/or animal blood circulation is maintained at body temperature or is increased or decreased depending on the requirements. The filter unit ensures that particles which could have passed from the device into the blood, blood substitutes, or the solutions to be introduced into the human and/or animal blood circulation, or excess gas from the blood, blood substitutes, or the solutions to be introduced into the human and/or animal blood circulation are separated before the blood, blood substitutes, or the solutions to be introduced into the human and/or animal blood circulation are returned to the patient. The pump ensures the continuous transport of blood, blood substitutes or the solutions to be introduced into the human and/or animal blood circulation from the patient to the device and again back to the patient. The device with a triple functionality comprises two additional pumps, which are responsible for the discharge of the filtrate and the supply of replacement fluid.

The devices operate extracorporally, which means that blood is taken from a patient continuously, the cleaning of blood and/or gas exchange and/or fluid exchange takes place outside the patient in one of the devices and the treated blood is continuously fed to the patient.

The semipermeable membrane is essentially permeable to electrolytes, urea, creatinine, phosphate, amino acids, medicaments and water.

The gas-permeable membrane of the device with dual functionality is essentially permeable to oxygen and carbon dioxide, but also for other gases. The gas-permeable membrane is not permeable to liquids. The gas-permeable membrane and the semipermeable membrane will be shortly referred to in the following as a membrane. The membrane may be present as a laminar film or a stack of films or as one or more bundles of hollow fibers. The membrane or rather the hollow fibers are made of a material or polymer selected from the group of: polyolefins, polyethylene (HDPE, LDPE, LLDPE), fluorinated polyethylene, copolymers of ethylene with butene-(1), pentene-(1), hexene-(1), copolymers of ethylene and propylene, EPR or EPT gum elasticum (third component with diene structure including dicyclopentadiene, ethylidennorbornene, methylendomethylenhexahydronaphthaline, cis-cis-cyclooctadiene-1,5-hexadiene-1,4), hexyo-(1-hexene methylhexadiene), ethylene-vinyl acetate copolymer, ethylene-methacrylic acid copolymer, ethylene-N-vinylcarbazole, methacrylamide-N,N′-methylene-bis(meth)acrylamide-allyl glycidyl ether, glycidyl(meth)acrylate, polymethacrylate, polyhydroxymethacrylate, styrene-glycidyl methacrylate copolymers, polymethyl pentene, poly (methyl methacrylate methacryloylamidoglutaminicacid), poly (glycidyl methacrylate-co-ethylene dimethacrylate), styrene-polyvinylpyrrolidone glycidyl methacrylate copolymer, polyvinylpyrrolidone blends with crospovidone, ethylene trifluoroethylene, polypropylene, polybutene (1), poly-4-(methylpentene) (1)), polymethylpentane, polyisobutylene copolymer, isobutylene-styrene copolymer, butyl gum elasticum, polystyrene and modified styrene, chloromethylated styrene, sulfonated styrene, poly-(4-aminostyrene), styrene-acrylonitrile copolymer, styrene-acrylonitrile-butadiene copolymer, acrylonitrile-styrene-acrylic ester copolymer, styrene-butadiene copolymer, styrene-divinylbenzene copolymer, styrene-maleic anhydride copolymer, polydienes in the cis-trans, in the 1-2 and 3-4 in the configuration, butadiene, isoprene, purified natural gum elasticum, Chloroporem, styrene-butadiene copolymer (SBR), triblockpolymer (SBS), NBR acrylonitrile-butadiene copolymer, poly-(2,3-dimethylbutadiene), a triblock copolymer terminated from polybutadien with cycloaliphatic secondary amines, or benzal-L-glutamate or polypeptides, or N-carbobenzoxylysin, poly-(alkenamere)polypentenamer, poly-(1-hexebmethyl-hexadiene), poly-phenylene, poly-(p-xylylene), polyvinyl acetate, vinyl acetate-vinyl copolymer, vinyl acetate-vinyxl pivalate copolymer, vinyl acetate-vinyl chloride copolymer, polyvinyl alcohol, polyvinyl formal, polyvinyl butyral, polyvinyl ethers, poly-(N-vinylacarbazol), poly-N-vinylpyrrolidone, poly-(4-vinylpyridine), poly-(2-vinylpyridiniumoxid), poly-(2-methyl-5-vinylpyridine), butadiene-(2-methyl-5-vinylpyridine) copolymer, polytetrafluoroethylene, tetrafluoroethylene-hexafluoropropylene copolymer, tetrafluorethylen-perfluoropropylvinylether copolymer, tetrafluoroethylene-ethylene copolymer, tetrafluoroethylene-trifluornitrososmethan copolymer, tetrafluoroethylene-perfluoromethylvinylether copolymer, tetrafluoroethylene-(perfluoro-4-cyanobutylvinylether) copolymer, poly-(trifluorchlormethylen, trifluorochloroethylene-ethylene copolymer, polyvinylidene fluoride, hexafluoroisobutylene-vinylidene fluoride copolymer, polyvinyl fluoride, polyvinyl chloride, chlorinated PE, FVAC or polyacrylates, soft PVC, post-chlorinated PVC, polyvinyl chloride-vinyl acetate copolymer, vinyl chloride-propylene copolymer, polyvinylidene chloride-vinyl chloride-vinyl chloride-vinylidene chloride copolymer, vinylidene chloride-acrylonitrile copolymer, polyacrylic acid, acrylic acid-itaconic acid copolymer, acrylic acid-methacrylic acid copolymer, acrylic acid ester-acrylonitrile copolymer, acrylic acid ester-2-chlorethylenvinylether copolymer, poly (1,1-dihydroperfluor-butyl acrylate), poly-(3-perfluormethoxy-1,1-dihydroperfluorpropylacrylat), polysulfone, polyacrolein, polyacrylamide, acrylic acid-acrylamide copolymer, acrylamide-copolymer maleic acid, acrylamide hydroxymethyl methacrylate copolymer, acrylamid methyl methacrylate-acrylamide copolymer, acrylamide-methyl acrylate copolymer, acrylamide-maleic anhydride copolymer, acrylamide-methacrylic acid copolymer, acrylamide-anilino-acrylamide copolymer, acrylamide-(N-4-acrylolcarboxymethyl-2,2-dimethylthiazoline) copolymer, polymethacrylic, methacrylic acid methacrylonitrile copolymer, methacrylic acid-3-fluoro styrene copolymer, methacrylic acid-4-fluoro styrene copolymer, methacrylic acid-3-fluoranilid copolymer, nitrated copolymers of methacrylic acid with methacrylic acid-3-fluoroanilid or fluorostyrene or copolymers of methacrylic acid with 3,4-isothiocyanatostyrene, or N-vinylpyrrolidone with maleic anhydride, or polyvinyl alcohol and polyallyl, polyacrylonitrile, acrylonitrile-2-vinylpyridine copolymer, acrylonitrile-methallyl sulfonate copolymer, acrylonitrile-N-vinylpyrrolidone copolymer, hydroxyl PAN, acrylonitrile-vinyl acetate copolymer, acrylonitrile-acrylic ester copolymer, polyallyl compounds, polydiallylphthalate, polytrisallylcyanurat, poly cyanoacrylate-α, polydimethylaminoethylmethacrylat and copolymer with acrylonitrile, methylmethacrylatlaurylmethacrylat copolymer, P-acetaminophenylethoxymethacrylat-methyl methacrylate copolymer, glycoldimethylmethacrylat methacrylate copolymer, poly-2-hydroxyethyl methacrylate, 2-hydroxymethylmethacrylate-methylmethacrylat copolymer, glycol dimethacrylate methacrylate copolymer, poly-2-hydroxymethylmethacrylat, 2-hydroxymethylmethacrylat-methylmethacrylat copolymer, glycolmethacrylat-glycoldimethylmethacrylat copolymer, styrene-hema-block and graft copolymers, poly-N, N-P, P-oxydiphenylenmellitimid, polydiethylenglycolbisallylcarbonat, aliphatic polyethers, polyoxymethylene, polyoxyethylene, polyfluoral, polychloral, polyethylene oxide, polytetrahydrofuran, polypropyleneoxide, ethylenoxydpropylenoxide copolymer, propylene oxide-allyl glycidyl ether copolymer, polyepichlorohydrin, ethylene oxide-epichlorohydrin copolymer, poly-1,2-dichloromethyl-ethyleneoxide, poly-2,2-bis-chloromethyl oxacyclobutan, epoxy resins, bisphenol-A diglycidyl ether, epoxidized phenol-formaldehyde, cresol-formaldehyde, resins, networking with anhydrides, amines such as diethylentriamin, isophorondiamide, 4,4-diaminodiphenyl methane, aromatic polyethers, polyphenylene oxides, polyphenol, phenoxy resins, aliphatic polyesters, polylactide, polyglycolide, poly-β-propionic acid, poly-β-D-hydroxybutyrate, polypivolactone, poly-ε-caprolactone, polyethylenglycoladipate, polyethylenglycol sebacate, unsaturated polyester from maleic anhydride, phthalic anhydride, isophthalic acid, terephthalic acid or HRT with, ethylene glycol, 1,2-propylene glycol, neopentyl glycol, ethoxylated bisphenols cyclododecandiol networking or unsaturated polyester resins or vinyl ester resins by copolymerization of unsaturated polyesters with styrene, methacrylate, vinyl monomers, vinyl acetate, methyl methacrylate, polycarbonate of bisphenol A and its derivatives and polyethers, polyesters, segmented polycarbonates from bisphenol A and its derivatives and aliphatic polyether, and aliphatic polyesters (see above), polyethylene glycol terephthalate (PET) surface-modified grafted with acrylic acid or by partial hydrolysis of the surface of PET, polyethylene glycol, polyethylene glycol adipate, polyethylene glycol terephthalate segmented, with polyether and aliphatic polyester blocks and polytetrahydrofuran blocks, poly-p-hydroxybenzoate, hydroxybenzoic hydroquinone copolymer, hydroxybenzoic acid-terephthalic acid copolymer, hydroxybenzoic-p, p-diphenyl ether copolymer, polyvinyl alcohol, polyvinyl pyrrolidone-maleic anhydride copolymer, alkyd resins from glycerol, pentaerythritol, sorbitol, with phthalic acid, succinic acid, maleic acid, fumaric acid, adipic acid and fatty acids from linseed oil, castor oil, soybean oil, coconut oil, aliphatic polysulfides (R—Sx—)=degree of sulfur, aromatic polysulfides, polythio-1,4-phenylene, and thiophene aromatic polysulphide of phenol, polyethersulfones, polysulfo-1,4-phenylene, poly-p-phenylensulfone, polyimines, polyethylenimine, polyethylene imines, branched polyethylene imines, polyalkylene amines, polyamides, polyhexamethylene adipamide, polyhexamethylene sebacamide, polyhexamethylendodekandiamide, polytridekanbrassylamide, versamide from vegetable oils with diamines and triamines, polyamide of ω-amino carboxylic acids with α-, β-, γ-, δ-aminocarboxylic acids or lactams, terephthalic acid m-aminobenzamide copolymer, terephthalic acid phenylenediamine copolymer, polyamidhydrazide e.g. from isophthalic acid and m-aminobenzhydrazide, polypiperazinamide, for example, fumaric acid and dimethyl piperazine, polybenzimidazoles from terephthalic acid and tetraaminobenzene (substituted), or from diaminodiphenyl ether and dichlorodiphenyl (substituted and cyclised) or from m-phenylene isophthalamide and terephthalamide, polyimides for example from pyromellitic dianhydride, methoxy-m-phenylenediamine, pyrones e.g. from pyromellitacidmedianhydride and diaminobenzidine, aromatic polyamides, poly-m-phenylenisophtalamide, poly-p-benzamide, poly-p-phenylenerephthalamid, m-aminobenzoic acid p-phenylendiamine isophthalsen copolymer, poly-4,4-diphenylsulfonterephthalamid from terephthalic acid and hexamethylenetetramine, terephthalic acid and mixtures of 2,4,4-trimethyl hexamethylene diamine-and 2,4,4-trimethyl hexamethylene diamine, from terephthalic acid, diaminomethylennorbornene and ε-caprolactam, from isophthalic acid and lauric lactame, from isophthalic acid and di-4-(cyclohexylamino-3-methyl)-methane, from 1,12-decandiacid and 4,4-diamino-dicyclohexylmethane, aromatic polyamides with heterocyclic compounds from dicarboxylic acid, terephthalic acid and isophthalic acid, diamin containing heterocycles with oxdiazole, triazole bithiazole and bezimidazol structures, 3-(p-aminophenyl)-7-amino-2,4-(1H,3H) quinazolinedione and isophthalic acid, polyamino acids, polymethyl-L-glutamate, poly-L-glutamic acid include copolypeptides, e.g. glutamic acid and leucine, phenylalanine and glutamic acid, glutamic acid and valine, glutamic acid and alanine, lysine and leucine, p-nitro-D, L-phenylalanine and leucine among others, polyureas from diamines and diisocyanates with ureas, polyurethanes from aliphatic and aromatic diisocyanates, and difunctional and trifunctional hydroxylated aliphatic polyesters and aliphatic polyethers and possibly modification with bifunctional amino, hydroxyl and carboxyl containing substances, such as hexamethylene diisocyanate, diphenylmethandiisocyanate, toluene diisocyanate, 2,4- and 2,6-tolidine diisocyanate, xylylenediisocanat, glycerin, ethylene glycol, diethylene glycol, pentaerythritol, 3-dimethyl-12-propanediol and carbohydrates, aliphatic and aromatic dicarboxylic acids and their derivatives, o-, p-, m-phenylenediamine, benzidine, methylene-bis-o-chloroaniline, p,p-diaminodiphenylmethane, 1,2-diaminopropane, ethylenediamine, amino resins from urea and cyclic ureas, melamine, thiourea, guanidine, urethane, cyanamide, amides and formaldehyde and higher aldehydes and ketones, silicones, polydialkylsiloxane diaryl siloxanes and aryl siloxanes such as alkyl dimethyl, diethyl-, dipropyl-, diphenyl-, phenylmethyl siloxane, silicone with functional groups, such as allyl, γ-substituted fluorinated silicones having amino groups and vinyl groups, such as from aminopropyltriethoxysiloxan, 2-carboxylpropylmethylsiloxan, block polymer with dimethylsiloxane and polystyrene or polycarbonate blocks, tri-block copolymers of styrene, butyl acrylate with α, ω-dihydroxy polydimethylsiloxane, 3,3,3-trifluorpropylmethylsiloxane, avocane (90 silicone and polycarbonate), hydrophobic polymers with the addition of hydrophilic polymers, such as polysulfone-polyvinylpyrrolidone, cellulose and cellulose derivatives, such as cellulose acetate, perfluorbutyrylethylcellulose, perfluoracetylcellulose, polyaromatic polyamide polymers, cellulose nitrate, carboxymethylcellulose, regenerated cellulose, regenerated cellulose from viscose, and similar cellulose derivatives, agarose, polysaccharides such as carrageenans, dextrans, mannans, fructosan, chitin, chitosan-(ethylene glycol diglycidyl ether) (chitosan-EGDE), chitosan, pectins, glycosaminoglycans, starch, glycogen, alginic acid, and all deoxypolysaccharide and their derivatives, murein, proteins, such as albumin, gelatin, collagen I-XII, keratin, fibrin and fibrinogen, casein, plasma proteins, milk proteins, crospovidone, structural proteins from animal and plant tissues, soy proteins, proteins from the food industry.

Additional materials or polymers are obtained by co-polymerization of the above-mentioned polymers, which are synthesized from different monomer units, with other monomers as listed in “functional monomer”, Ed. R H. Yocum and E. B. Nyquist, Vol I and II, Marcel Dekker, New York, 1974. Furthermore, the above polymers can be modified partially or fully by grafting and by producing further block copolymers and graft copolymers. In addition, polymer blends, coated polymers and polymers can be produced in the form of various composite materials. Furthermore, polymer derivatives can be prepared with bi-and polyfunctional cross-linking reagents as they are known of the methods of peptide, protein, and polysaccharide and polymer chemistry for the production of reactive polymers.

Thereby hydrophobic polymers are preferred. Particularly preferred are membranes or hollow fibers consisting of the following materials or polymers: silica, silicones, polyolefins, polytetrafluoroethylene, polyesterurethane, polyetheruretane, polyuerethane, polyethylene terephthalate, polymethylpentane, polymethylpentene, polysaccharides, polypeptides, polyethylenes, polyesters, polystyrenes, polyolefins, polysulfonates, polypropylene, polyethersulfones, polypyrroles, polyvinylpyrrolidone, polysulfones, polylactic acid, polyglycolic acid, polyorthoesters, polyaromatic polyamide, aluminum oxide, glass, sepharose, carbohydrates, copolymers of acrylates or methacrylates and polyamides; polyacrylic ester, polymethacrylic ester, polyacrylamide, polymethacrylamide, polymethacrylate, polyetherimide, polyacrylonitrile, copolymers of ethylene glycol diacrylate or ethylene glycol dimethacrylate and glycidyl acrylate or glycidyl methacrylate and/or allyl glycidylether, regenerated cellulose, cellulose acetate, hydrophobic polymers with the addition of hydrophilic polymers, derivatives and copolymers of these polymers.

The length of the hollow fibers is between 30-150 mm, preferably between 50 and 100 mm. The outer diameter of such a hollow fiber is about 0.1-1.5 mm, the inner diameter is approximately 0.1-1 mm while the wall thickness of the membrane or hollow fiber itself is 5-200 μm, preferably 15-50 μm.

The walls of the hollow fibers may comprise pores. The porosity of the inner and outer surface of hollow fibers of the gas-permeable membrane is in the range of 10 to 90%. The pores have a diameter in the range of 0-5 μm, and preferably have a diameter of from 0 to 1.5 μm. Generally, the pore size should be kept as low as possible, since during prolonged use of the device with dual functionality undesirable plasma can penetrate through the pores into the chamber were the gas is flowing through and thus is withdrawn from the patient which also leads to a decrease in performance of the device with dual functionality. The pores in the fiber walls are preferably formed by stretching or by solid-liquid phase separation.

The porosity of the inner and outer surface of the hollow fibers of the semipermeable membrane is in the range of 10 to 90%. The pores preferably have a diameter in the range of 0.01 to 1.5 μm.

The hollow fibers of the membrane have an inner and an outer surface. The inner surface represents the surface of the lumen of the hollow fibers and the outer surface is the surface of the outer surface of the hollow fibers. The entire surface of the hollow fibers is between 0.1 and 6 m².

The carrier, which is coated with substances for adsorptive removal of toxins of biological and chemical synthetic origin, their metabolites and degradation products in blood, blood substitutes or solutions for the introduction into the human and/or animal blood circulation, can take the form of particles or the form of hollow fibers. If the carrier is provided in the form of hollow fibers, the carrier and the gas permeable membrane are combined to a unit or rather form a unit or together form an inseparable component. The carrier in the form of hollow fibers includes all the aforementioned properties of the gas permeable membrane. In this case the carrier in the form of hollow fibers fulfills two functions. On the one hand it ensures a gas exchange, preferably an exchange of oxygen and carbon dioxide between the current of blood, the blood substitute or solutions for the introduction into the human and/or animal blood circulation on one side of the hollow fiber and the gas flow on the other side of the hollow fiber. Moreover it is also coated with substances for adsorptive removal of toxins of biological and chemical synthetic origin, their metabolites and degradation products in blood, blood substitutes or solutions for the introduction into the human and/or animal blood circulation. Thus, the carrier fulfills a second function, namely the simultaneous binding and thereby removal of toxins of biological and chemical synthetic origin, their metabolites and degradation products from blood, blood substitutes or solutions for the introduction into the human and/or animal blood circulation.

As an alternative embodiment, the carrier can be provided in the form of particles. The particles are also composed of polymers. Hereby, independently from the hollow fibers, polymers for the particles are selected from the same group of polymers, as listed for the hollow fibers. The following polymers are preferred for particles: methacrylamide-N, N′-methylene bis (meth) acrylamide-allyl glycidyl ether, glycidyl methacrylate, polyacrylic acid, dextran, regenerated cellulose, cellulose, polysaccharide, polymethacrylate, polyhydroxymethacrylate, polysulfone, polyethersulfone, styrene-glycidyl methacrylate copolymers, styrene-glycidyl methacrylate-polyvinyl copolymers, silicones, styrene-maleic anhydride copolymer, crospovidone (popcorn polymers), styrene-polyvinylpyrrolidone blends with crospovidone, zeolites, MCM's (Mm/x[AlmSinO₂(m+n)] pH₂O), polyamides, polyhydroxymethacrylate, poly (methyl methacrylate methacryloylamidoglutaminic acid), chitosan (ethylene glycol diglycidyl ether) (chitosan-EGDE), chitosan, poly (glycidyl methacrylate-co-ethylene dimethacrylate), polyvinyl alcohol, polyacrylamide.

The particles may be provided in the following forms: spherical, cylindrical, irregular, circular. The particles have a diameter of 50 μm-5 mm. The inner diameter of the circular particles is between 20 μm-4.5 mm. Due to their size and shape the particles are able to form packages in the column of the device, which contain channels that are permeable for the components of blood and whole blood, especially for the blood cells. Clogging of the particle packing in the column is avoided in this way. The particles also have an outer surface.

Furthermore, the carrier may have pores, either when provided as particles or when provided in the form of hollow fibers or hollow fiber bundles. In case the carrier is provided as a hollow fiber, the pores are present in its walls and pass essentially completely through the walls, so that the pores form channels between the inside (the lumen side) and the outside of the hollow fibers. Through these channels oxygen and carbon dioxide molecules diffuse. Oxygen and carbon dioxide molecules can also diffuse directly through the walls of the hollow fibers.

The porosity of hollow fibers or particles ranges from 10 to 90%. The pores have a diameter in the range of 0-5 μm, and preferably have a diameter of 0 to 1.5 μm. The pores in hollow fibers or particles also have a surface, which is termed as the inner surface of the pores or as the surface of the pores.

The surfaces of the carriers have chemical functional groups that are either part of the polymer the carrier consists of, or which were prepared by the activation, modification or reaction with a crosslinking agent of the surfaces of the carriers.

The surfaces can be activated or modified by high-energy radiation, exposure to light, oxidation, hydrolytic extension, by photochemical reactions, plasma treatment, by halogenation, sulfochlorination, chloromethylation, esterification, etherification, epoxidation, by reaction with radical formers, amines, amides, imides, isocyanates, aldehydes, ketones, nitriles, vinyl compounds, carboxylic acids and derivatives, and diazo compounds.

As chemical functional groups or cross-linking molecules on the surface of the carrier the following may be considered: phosgene, formaldehyde, glyoxal, acrolein, glutaraldehyde, azides, activated esters, anhydrides, acid chlorides, esters, mixed anhydrides, cyanogen bromide, difluordinitrobenzene thioisocyanates, epoxies, imides, isocyanates, urethione groups, diisocyanates, tri-isocyanates, maleimide, dicyclohexylcarbodiimide, N,N-bis-(trimethylsilylsulfurdiimide), peroxides, vinylketon groups, aromatic diazo compounds, vinyl sulfones, trichlorotriazine, monochlorotriazine, dichlorotriazine, bromacrylamide, difluorchlorpyrimidine, trifluoropyrimidine, dichloroquinoxaline, chloracetylamino groups, chloracetylurea, β-halogenpropionamide, α,β-dihalogenpropionamide, β-quaternary ammoniumpropionamide, β-sulfatopropionamide, β-sulfonylpropionamide, substituted alkane-dicarboxamide, substituted alkane monocarboxylates, substituted cycloalkane-carboxamides, alkene monocarboxamide, arylamide, crotonamide, substituted acrylamides, mono-, di-and trihaloarylamides, substituted crotonamide, alken-dicarboxamide, cyclic halogenmaleinimide, alkyne carboxamides, substituted aliphatic ketones, amides of substituted aliphatic ketones, amides of substituted aliphatic sulfonic acids, substituted methanesulfonamide, substituted ethansulfonamide, β-thiosulfatoethylsulfonamide, quaternary ammoniummethansulfonamide, vinylsulfonamide, β-chlorvinylsulfonamide, esters of reactive aliphatic sulfonic acids, β-substituted ethylsulfonic, β-thiosulfatoethylsulfone, β-halogenvinylsulfone, β-substituted ethylaminderivates, β-sulfatoethylamine, β-halogenethylpyrazolone, N-(β-halogen-ethyl)-amide, N-(β-sulfatoethyl)-amide, β-substituted ethylammonium compounds, β-substituted ethylamides of sulfonic acid, N,β-halogenethylsulfonamide, β,γ-dihalogenpropionylamide of sulfonic acids, β-sulfatoethylamide of sulfonic acids, ethylenimine and ethylenimine compounds, allyl groups, propargyl groups, diallyl phthalate, triallylcyanurate, benzyl derivates, 2-substituted thiazolcarbonacids, chlorsulfonylpyridine, 4-substituted 3,5-dicyano-2,6-dichloropyridine, 2,6-bis-(methylsulfonyl)-pyridine-4-carbonyl chloride, chlorpyridazine, dichlorpyridazone, 1-alkyl-4,5-dichloro-6-pyridazone, chlorine and bromopyrimidine, 3-(2,4,5-trichloropyrimidyl(6)amino)aniline, 4,5,6-trichloropyrimidine-2-carbonyl chloride, trifluoropyrimidine, 2-chlortriazinylderivates, 2-chloro-4-alkyl-s-(6-trizinyl-6-amino carboxylic acid), 2-chlorobenzothiazolcarbonyl, 6-amino-2-fluorbenzothiazoi, 2-methylsulfonyl-6-aminobenzothiazole, 2,3-dichloroquinoxaline-6-carbonyl chloride, 1,4-dichlorphthalazin-6-carbonyl chloride, 3-chloro-1,2,3-benzotriazine-1-N-oxide-7-carbonyl chloride, fluorine-2-nitro-4-azidobenzene, sulfonic acid, N-sulfonylureas, thiosulfato S-alkyl, N-methylthylolureas, N,N-dimethylol-glyoxal-monourein, terephthaldialdehyde, mesitylentrialdehyde, isothiuronium groups, triacylformal, 4-azido-1-fluoro-2-nitrobenzene, N-(4-azido-2-nitrophenyl)-1,1-aminoundecanoic and oligomethacryl acid.

Preferred chemical functional groups are primary amines, which can be converted with carbonyl compounds to imines and which can be optionally converted by hydrogenation to a stable amine bond afterwards. In addition carboxylic acids can be immobilized at amines via an amide bond. The usage of aziridines, oxiranes, maleinimides, N-succinimidylesters, N-hydroxysuccinimides, hydrazides, azides, aldehydes, ketones, carboxylic acids, carboxylic acid esters and epoxides is preferred.

The chemical functional groups are used to immobilize the substances for adsorptive removal of toxins of biological and chemical synthetic origin, their metabolites and degradation products on the surfaces of the carrier. The substances for adsorptive removal of toxins of biological and chemical synthetic origin, their metabolites and degradation products in blood, blood substitutes or solutions for the introduction into the human and/or animal blood circulation will be shortly referred to in the following just as substances.

The following combinations of the various surfaces of the carriers are coated specifically with the substances:
Carrier in the form of hollow fibers: outer surface or surface of the lumen
Carrier in the form of hollow fibers: outer surface and surface of the lumen
Carrier in the form of particles: outer surface of the particles
Carrier in the form of particles: outer surface and surface of the pores

The coated surfaces are those surfaces of the carriers which come in direct contact with the blood, blood substitutes, or the solutions to be introduced into the human and/or animal blood circulation.

The immobilization of the substances is conducted preferably covalently. A different bonding, for example, by hydrophobic, electrostatic and/or ionic interactions is also possible. The substances can be bound directly to the surfaces of the carrier by the chemical functional groups.

Alternatively, so-called linker molecules also known as spacers or crosslinking agents can be bound to the chemical functional groups on the surfaces of the carriers. These elongated linear molecules have at each end a reactive functional group. One of these ends can be bound specifically to the chemical functional groups on the surfaces of the carriers. The other end with its functionality is available for binding the substances to the surfaces of the carrier. Thus, the substances can be bound via linkers to the surfaces of the carriers. The described linear compounds can be used as a linker, wherein the reactive functionality is also selected from the group of said chemical functional groups. The ability to react and to form a bond with the existing chemical functional groups on the surface of the carriers is critical for the selection of a suitable reactive functionality. Alternatively, the linker can be selected from one of the mentioned cross-linking molecules.

The substances for adsorptive removal of toxins of biological and chemical synthetic origin, their metabolites and degradation products in blood, blood substitutes or solutions for the introduction into the human and/or animal blood circulation are selected from the following group of substances: polyacrylic acid and derivatives of polyacrylic acid, albumin, metal chelate complexes, cyclodextrins, ion exchangers, linear and cyclic poly- and oligoamino acids, modified polyamino acids, modified and unmodified polyethylenimine, polyallylamine and modified polyallylamine, basic oligopeptides, immobilized amidine groups, histidine, polypropylene, polyethylene, polyvinylidene fluoride, polytetrafluoroethylene, alkylaryl groups, monoaminoalkane, toxic shock syndrome toxin 1-binding peptides (toxic shock syndrome toxin 1-binding peptide, TSST 1-binding peptides), diaminoalkanes, polyaminoalkane, aromatic nitrogen-containing heterocyclic compounds and their derivatives, antimicrobial peptides (AMP), endotoxin-neutralizing protein (endotoxin neutralizing protein, ENP), synthetic peptides, polylysine, HDL, cholesterol, polymyxin B and polymyxin E (colistin), membrane-forming lipids and lipoproteins and polysaccharides and lipopolysaccharides, glycoproteins, cholesterol esters, triacylglycerols, in general steroids, phosphoglycerides, sphingolipids, lipoproteins with and without cyclic portion, lipooligosaccharides with protein content, peptides having the formula R-(Lys-Phe-Leu)ₙ-R₁ with R and R₁=H or wherein R is an amino protecting group or H and R₁ is a carboxy protecting group or H, amino acid residues, fatty acid residues in length between 1-100 carbon atoms, preferably 1-10 carbon atoms; nitrogen-containing heterocyclic compounds, nitrogen-functionalized aromatic carboxylic acids and/or their derivatives. Heparin, heparin derivatives, heparan, heparan derivatives, oligosaccharides and polysaccharides and preferably oligosaccharides and polysaccharides containing iduronic acid, glucuronic acid, glucosamine, galactosamine are less or not preferred for endotoxine adsorption and therefore are not or not preferably used for toxin adsorption.

Preferred materials for adsorptive removal of toxins of biological and chemical synthetic origin, their metabolites and degradation products in blood, blood substitutes or solutions for the introduction into the human and/or animal blood circulation are albumin, synthetic peptides, polylysine, lipoproteins with and without a cyclic residue, lipooligosaccharides with protein content, antimicrobial peptides (AMP), HDL, cholesterol, endotoxin-neutralizing protein and toxic shock syndrome toxin 1-binding peptides (toxic shock syndrome toxin-1-binding peptides, TSST-1-binding peptides).

For selective coating of the outer surface of the carrier in hollow fiber form or rather for targeted immobilization of substances for adsorptive removal of toxins of biological and chemical synthetic origin, their metabolites and degradation products, on this surface, at first the pores and then the inner space, the lumen, the carrier is filled with a medium. Under the conditions of filling, the media is liquid and therefore completely covers the surface of the lumen and the pores. In addition, this media is not miscible with a solution which is used afterwards for coating the outer surface of the carriers in hollow fiber form. Due to the fact that the medium completely covers the surface of the lumen as well as the inner surface of the pores and is not miscible with the solution for coating of the outer surfaces of the carriers, the solution for coating of the outer surfaces of the carriers cannot coat the surfaces of the lumen or the inner surfaces of the pores, so that coating takes place only on the outer surfaces of the carriers in hollow fiber form. After coating the outer surfaces of the carrier in hollow fiber form, which proceeds preferably completely or quantitatively, the medium is removed from the lumen and the pores of the carrier.

For targeted coating of the surface of the lumen of the carrier in hollow fiber form the pores are initially filled with the medium. Then the lumen of the carrier is filled with the solution that is used for coating the surface of the lumen in hollow fiber form. After coating the surfaces of the lumen, which proceeds preferably completely or quantitatively, the medium from the pores of the carrier and the solution from the lumen of the carrier are removed.

The outer surface and the surface of the lumen of the carrier in hollow fiber form can be coated similarly by first filling the pores with the medium and then filling the lumen with the solution for coating and then surrounding the outer surface of the carrier with the solution for coating. Linear, branched, acyclic or cyclic C₁-C₂₀ alkanes such as hexane, heptane or dodecanol can be used as medium.

A carrier in hollow fiber form is obtained, which is only coated on its outer surface, while the surfaces of the lumen remain uncoated. Or a carrier in hollow fiber form is obtained, which is only coated on the surfaces of its lumen, while its outer surfaces remain uncoated. Hence, by filling the lumen and the pores of the carrier, specific surfaces of the carriers could be protected from certain coatings.

For coating of the outer surface of the carrier in the form of particles as well as the inner surfaces of its pores or rather for immobilization of the substances for adsorptive removal of toxins of biological and chemical synthetic origin, their metabolites and degradation products on these surfaces, the particles are suspended in the coating solution and the pores are also filled thereby. After coating of the outer surfaces of the carrier in particle form as well as the inner surfaces of its pores, which proceeds preferably completely or quantitatively, the solution is removed from the particles and out of the pores of the carrier.

Due to the specifically coated and non-coated surfaces the obtained carriers exhibit different properties on these surfaces respectively.

The carriers in hollow fiber form are either coated with substances on the outer surface or on the surface of the lumen, so that the outer surface or the surface of the lumen is washed around by blood, blood substitutes or solutions for the introduction into the human and/or animal blood circulation. The respective other surface of the carrier in hollow fiber form remains uncoated. Since the carrier is preferably made out of hydrophobic polymers, the respective uncoated surface has hydrophobic properties. Over this uncoated surface the gas stream is guided.

Thus, in a carrier in hollow fiber form, the uncoated surface over which the gas stream is guided lies opposite to the substance coated surface, which is in contact with blood, blood substitutes or solutions for the introduction into the human and/or animal blood circulation. Both surfaces are connected by the pores, which lead through the wall of the hollow fiber carriers. The toxins of biological and chemical synthetic origin, their metabolites and degradation products adsorb to the substances that are either coated to the lumen or the outer surface of the carrier and are retained on these surfaces. The toxins of biological and chemical synthetic origin, their metabolites and degradation products are thereby removed from the blood, blood substitutes, or the solutions to be introduced into the human and/or animal blood. In case of blood, the therein contained carbon dioxide diffuses through the pores of the carrier into the space through which gas flows and is removed by the gas stream. At the same time the small diameter of the pores, the hydrophobic properties of the gas stream contacting surface and the inner surface of the pores prevent the blood stream from also passing through the pores into the space through which gas flows.

The diameter of the pores is chosen so that it is smaller than the diameter of a blood cell. A pore size of ≦1.5 μm is preferred, more preferred is a pore size of ≦1.0 μm because the maximum pore size of 1.5 μm is smaller than the smallest blood cells, which have a diameter of about 2 μm. The blood cells therefore can not penetrate into the pores of the carrier.

The oxygen contained in the gas stream can also diffuse through the pores of the carrier and so enters the space through which blood flows. This results in an enrichment of blood with oxygen. By this way, toxins of biological and chemical synthetic origin, their metabolites and degradation products as well as carbon dioxide can be removed from the blood and oxygen is enriched in blood at the same time.

As discussed above, a trespass of the blood stream through the pores of the carrier into the space through which gas flows is prevented, amongst others by the hydrophobic properties of the surface of the carrier in the form of hollow fiber which is in contact with the gas stream and the inner surface of the pores. In devices which carry out only gas exchange in blood, plasma leakage, i.e. trespassing of, for instance, blood plasma into the compartment through which the gas flows, is a frequent and serious problem that hinders the gas exchange. By comparing the device with dual functionality to a device that only performs gas exchange in blood it has been surprisingly found that plasma leakage occurs only very rarely and the gas exchange is reliable without hindrance and with high gas transfer rates.

The inner surface or the outer surface of the carrier in the form of hollow fibers, or the outer surface of the carrier in the form of particles can also exhibit a hemocompatible coating additionally to the substance coating. The hemocompatible coating is applied in each case on the side of the hollow fibers, which will come in contact with blood, blood substitutes or solutions for the introduction into the human and/or animal blood circulation. The hemocompatible coating prevents or reduces responses of blood to the surfaces of the device, which are recognized as foreign, and therefore results in a more gentle treatment of the patient. It has surprisingly been found when coating the surface of carriers with substances and additionally a hemocompatible coating both coatings retain their full functions without impeding each other. The substance coating of the carrier surface serves the function of removal of toxins of biological and chemical synthetic origin, their metabolites and degradation products of blood, blood substitutes or solutions for the introduction into the human and/or animal blood circulation. The function of the hemocompatible coating persists in the prevention or reduction of responses of the blood to the foreign surfaces of the carrier and the device. There was reason for concern that the additional hemocompatible coating on the carrier surfaces could lead to a change of their surface properties, which would adversely affect the interaction of the substances on the carrier surface with toxins of biological and chemical synthetic origin, their metabolites and degradation products and so could reduce the binding capacity of the substances for said toxins. Surprisingly, this concern was not confirmed.

The hemocompatible coating consists of heparin or chemically modified polysaccharides, i.e. chemical derivatives of the polysaccharides. The chemical modifications of polysaccharides comprise desulfation, resulfation, deacylation and/or reacylation to various extents.

The polysaccharides are selected from the group of: glycosaminoglycans, synthetic oligo- and polysaccharides, glucosaminoglycans, chemically modified heparin and heparan sulfate, chondroitin sulfate, dermatan sulfate, keratan sulfate, hyaluronan, onuphinic acid, carrageenans, chitin, xylans, dextrans, mannans, xyloglucans, galactans, xanthan, arabinogalacturonans, rhamnogalacturonans, galaktomanans, pectins, amylopectins, lambda, agar-agar, agarose, algin, alginates, ghatti gum, gum arabic, tragacanth, karaja gum, locust bean gum, gua gum, tara gum, manucol, kelgine, pululan, isolichenin, Nigeran mycodextran, Elsinoe leucospila a-glycan, alternans, Evernia prunastri α-glycan, pustulan, icelandic acid, acid luteic, Microellobosporia mannoglucan, agrobacterium β-glucans, Rhizobium β-glucans, Acetobacter β-glucan, mycoplasma β-glucan, Escherichia coli (1-2)-β-oligoglucosides, curdlan, laminarin, paramylon, chrysolaminarine, cellulin, mycolaminarin, lichenin, callose, furcellaran, heparin, urokinase, HEMA-St-HEMA copolymer and poly-HEMA and its chemical derivatives.

Such hemocompatible coating is optional and also preferred in only a few cases wherein the substances used for the hemocompatible coating are not used for the adsorption of toxins and also not contribute to the adsorption of toxins.

Moreover, the inner surface or the outer surface of the hollow fibers can be coated with a surface tension reducing coating. The surface tension-reducing coating is applied in each case on the side of the hollow fibers, which will come into contact with blood. The reduction of surface tension leads to an efficient priming of the device.

Before the device is used on a patient, the entire inner space dedicated for the blood stream must be filled with liquid. This process is called priming and the necessary volume of liquid is the priming volume. The priming is necessary to completely remove unwanted gas from the blood-carrying inner space of the device, to wet the surface of the blood-carrying space and to ensure that the extracorporeal blood circulation is completely liquid-filled when the device is connected to the bloodstream of the patient. The term “blood-carrying inner space of the device” or the “blood-carrying space” refers to all spaces or surfaces of the device that come in contact with blood, blood substitutes, or the solutions to be introduced into the human and/or animal blood circulation. The blood-carrying inner space of the device or the blood-carrying space also includes the chamber of the device, which is flown through by blood, blood substitutes or solutions for the introduction into the human and/or animal blood circulation.

If wetting agents have been applied to the surfaces of the blood-carrying spaces of the device, advantageously they may be removed during the priming and hence support the priming process. The priming volume is dependent on the total volume of the blood-carrying inner spaces of the device. The larger the volume of blood-carrying inner spaces the greater is the volume of priming fluid, which mixes in the extracorporeal circuit with the blood circulation of the patient. The mixture of the priming solution with the bloodstream of the patient leads to hemodilution, which is an additional burden for the patient. Therefore it is advantageous to keep the priming volume as low as possible. The following solutions or a mixture thereof may be used as priming fluid: saline solution 0.9%, Ringer's lactate solution, HAES, mannitol, heparin, cortisone, sodium bicarbonate solution, tranexamic acid (formerly Aprotenin).

To reduce the surface tension, the surfaces of the hollow fibers can be coated with a wetting agent. Such wetting agents are amphoteric, zwitterionic, nonionic, anionic and/or cationic compounds. The wetting agents for the surface tension-reducing coating are selected from the group of the following compounds: Amphoteric wetting agents comprise for example lauroamphocarboxyglycinate, e.g. MIRANOL 2MHT MOD available at Miranol, Inc. (Dayton, N.J.) or synergistic components thereof. Exemplary zwitterionic wetting agents comprise β-N-alkylaminopropionic acid, N-alkyl-β-iminodipropionic acid, fatty imidazoline carboxylate, N-alkyl betaines, sulfobetaines, sultaines and amino acids, e.g. asparagine, L-glutamine etc. examples for anionic wetting agents comprise aromatic hydrophobic esters and anionic fluorine-containing wetting agents. The cationic wetting agents include methyl-bis-hydrogenated talgamidoethyl, 2-hydroxyethylammoniummethylsulfate, water-soluble quaternized condensation polymers, cocoalkylbis-(2-hydroxyethyl)-methyl and ethoxylated chlorides. Non-ionic wetting agents comprise alkoxylated alkyl amines, ethanol, isopropanol, methanol, glycerol, alkyl pyrrolidones, linear alcohol alkoxylates, fluorinated alkyl esters including aminoperfluoroalkylsulfonate, N-alkyl pyrrolidone, alkoxylated amines and poly (methylvinylether/maleic anhydride) derivatives. Other wetting agents comprise oligomeric or non-monomeric compounds containing C12-18 aliphatic and/or aromatic hydrophobic residues and a hydrophilic functionality within the same molecule. Other wetting agents comprise difunctional block copolymers with terminal secondary hydroxyl groups and difunctional block copolymers with terminal primary hydroxyl groups. These block copolymers typically contain repeating units of poly (oxypropylene) or propylene oxides (POP) and poly(oxyethylene) or ethylene oxide (POE). Non-toxic and hemocompatible wetting agents are preferred.

The surface tension-reducing coating with wetting agent is applied reversible to the inner or outer surface of the hollow fibers, so that the wetting agent can be washed away from the surfaces before the surfaces come into contact with blood.

The devices are used to remove toxins from biological and chemical synthetic origin, their metabolites and degradation products in blood, blood substitutes or solutions for the introduction into the human and/or animal blood circulation. The toxins of biological and chemical synthetic origin, their metabolites and degradation products are selected from the group: fibrinogen, toxins associated with an infectious disease, toxins associated with nutrition, e.g. fungal toxins, nicotine, ethanol, botulism; toxins from work-related and from criminal acts e.g. lead acetate, B-and C-weapons; toxins in the form of gas, aerosol, liquid and solids such as CO; immune complexes, medicaments, drugs, alcohol, detergents, phosgene, chlorine, hydrogen cyanide, nitrosamines, oxalic acid, benzopyrenes, solanine, nitrates, nitrites, amines, dichlorodisulphide, halogenated hydrocarbons; toxins of bacterial, fungal e.g. mycotoxins as epoxytrichotecene, ochratoxin A, zearalenone; and protozoal origin and their components e.g. exotoxins, endotoxins, fungal spores; and their degradation products, biological warfare toxins such as microcystins, anatoxin, saxitoxin of bacterial origin and their degradation products, insecticides, bactericides, drugs and their metabolites, narcotics, pharmaceuticals and their metabolites and their degradation products, antigens, DNA, RNA, ENA, immunoglobulins, autoimmune antibodies, antibodies, including anti-DNA antibodies, anti-nuclear antibodies, viruses, retroviruses and viral components, such as hepatitis virus particles, lipids, proteins, peptides, proteolipids, glycoproteins and proteoglycans, fibrin, prions, nano weapons, metals, such as Hg, Cd, Pb, Cr, Co, Ni, Zn, Sn, Sb, and ions of these metals, semimetals, such as As; as well as ions of these semi-metals, toxic lipopolysaccharides and endotoxins.

Preferred are toxins of biological and chemical-synthetic origin, whose metabolites and degradation products are toxic endotoxins and lipopolysaccharides.

The endotoxins or toxic lipopolysaccharides can exemplarily originate from the following organisms: Escherichia coli, Salmonella, Shigella, Pseudomonas, Neisseria, Haemophilus influenzae, Bordetella pertussis and Vibrio cholerae.

Chemically the endotoxins correspond to lipopolysaccharides (LPS); LPS are amphipathic molecules. The hydrophobic part, the lipid A, contains five to seven saturated fatty acids bonded to a glucosamine dimer. The hydrophilic head of the LPS molecule consists of an oligosaccharide, the central core region and the O-antigen, a polymer of repeating units of three to six sugar residues, varying also within a bacterial membrane (neutral sugars with 5-7 carbon atoms, deoxy and amino sugars, uronic and amino-uronacids, O-methyl-, O-acetyl-, phosphate- and amino acid substituted sugar). The core region contains many negatively charged carbohydrate residues and phosphate residues and also binds divalent cations, hence creating a kind of permeability barrier.

On the basis of current knowledge it is believed that the pathophysiologic interactions depend on the toxically active part of endotoxin, the lipid A. It reacts with receptors on immune cells, mainly macrophages. Lipid A initially binds to the membrane-bound CD14 (cluster of differentiation). By a still unexplained mechanism of intracellular signal transduction the affected cells produce and then secrete inflammatory mediators (IL-1, IL-6, IL-12, TNF-α) and thus activate the immune system, including the humoral immune system.

As part of the response to the binding of lipid A the macrophages also release CD 14 in the surrounding area. These can also influence cells that usually do not respond to the lipid A. For example, endothelial cells express increasingly selectins and integrins after binding of CD14, which in turn causes an increased adhesion of leukocytes and platelets to the vessel walls.

The increased adhesion of platelets to the vessel wall leads to the activation of coagulation and release of kinins (e.g. bradykinin), resulting in the formation of clots that trigger the process of fibrinolysis in the course of their degradation. The kinin release also causes vasodilation.

In a nutshell, the effects of endotoxin disturb the balance between inflammation, coagulation and lysis. Possible consequences consist in inflammation, mediated by the action of mediators and activation of the complement system. Serious consequences include organ failure caused by disturbances in the microcirculation during thrombus formation and by shock due to vasodilation, as well as disseminated intravasal coagula through activation of coagulation and fibrinolysis.

Since the core region contains many negative charges, it interacts preferably with electrophilic or positively charged groups, such as with organic ammonium ions. This also explains the adsorption of LPS and endotoxins to nitrogen-containing compounds, such as antimicrobial peptides (AMP), endotoxin-neutralizing protein (endotoxin neutralizing protein, ENP), synthetic peptides, polymyxin B and polymyxin E (colistin), albumin, peptides having the formula R-(Lys-Phe-Leu)ₙ-R with R and R₁=H, amino acid residue, fatty acid group and n=1-100, preferably 1-10; polyethylenimine, polyamino acids, nitrogen-containing heterocyclic compounds with nitrogen groups of functionalized aromatic carboxylic acids and/or their derivatives.

The device is used for enrichment of blood, blood substitutes or solutions for the introduction into the human and/or animal blood circulation with oxygen and removal of carbon dioxide from the blood, blood substitutes or solutions for the introduction into the human and/or animal blood circulation. At a flow rate of the blood, blood substitutes or the solutions to be introduced into the human and/or animal blood circulation of 1 L/min the devices achieves an oxygen transfer rate of up to 100 ml/min and at a flow rate of the blood, blood substitutes or the solutions to introduced into the human and/or animal blood stream of 7 L/min, an oxygen transfer of up to 650 ml/min is achieved. The carbon dioxide transfer is up to 80 ml/min at a flow rate of the blood, blood substitutes or the solutions to be introduced into the human and/or animal blood circulation of 1 L/min and up to 350 ml/min at a flow rate of the blood, blood substitutes or solutions for the introduction into the human and/or animal blood circulation of 7 L/min.

The devices are used for the simultaneous removal of toxins of biological and chemical synthetic origin, their metabolites and degradation products and carbon dioxide from the blood, blood substitutes or solutions for the introduction into the human and/or animal blood circulation and for enriching the blood, blood substitutes or solutions for the introduction into human and/or animal blood circulation with oxygene. The removal of toxins of biological and chemical synthetic origin, their metabolites and degradation products, e.g. endotoxins, and carbon dioxide from the blood and the enrichment of blood with oxygen have proven to be an effective combination for prevention, alleviation or treatment of diseases. The device and method are thus used for prevention, alleviation or treatment of diseases caused by toxins of biological and chemical synthetic origin, their metabolites and degradation products.

The devices and method have been proven to be effective against diseases caused by the decay of gram-negative bacteria. The devices and method are thus for the prevention, alleviation or treatment of diseases, caused by the presence of lipopolysaccharides or endotoxins as membrane fragments of gram-negative bacteria.

The diseases which are caused by toxins of biological and chemical synthetic origin, their metabolites and degradation products or which can be attributed to the presence of lipopolysaccharides or endotoxins in form of membrane fragments of gram-negative bacteria are selected from the following group of diseases: endotoxemia, sepsis, fever, inflammation, organ failure, multiple organ failure, disseminated intravasal coagula, rhabdomyolysis, necrosis, shock, trauma, bacteremia, diarrhea, leukocytosis, vasodilation, coagulation due to hypotension, circulatory failure, systemic inflammatory response syndrome (systemic inflammatory response syndrome=SIRS), respiratory distress syndrome of adults (ARDS=acute respiratory distress syndrome), etc.

In particular, the combined use of these treatments is an effective way for prevention, alleviation or treatment of sepsis. Especially advantageous is the simultaneous application of said treatments, made possible by the devices, because the patient, severely weakend by sepsis, does not have to cope with two or three different treatments. Hence the treatment is not only very effective but also very gentle. Moreover, the simultaneous treatment saves precious time, which would have been lost otherwise by separate organ replacement therapy followed by treatment with endotoxin adsorption. For the hospital staff this simultaneous application creates no additional work and there is no longer the need to decide whether and when an endotoxin adsorption should be conducted. Hence, the decision-making process is removed and the sequential application of two or more treatments during the critical phase of a patient saves valuable time so that the risk of death for the patient due to sepsis is significantly reduced.

The devices are used for a method for removal of toxins of biological and chemical synthetic origin, their metabolites and degradation products in blood, blood substitutes or solutions for the introduction into the human and/or animal blood circulation, which comprises the following steps.
a) providing a device for removal of toxins of biological and chemical synthetic origin, their metabolites and degradation products out of blood, blood substitutes or solutions for the introduction into the human and/or animal blood circulation;
b) passage of blood, blood substitutes or solutions for the introduction into the human and/or animal blood circulation.

Here, the columns I and/or II, which are present in the device, can be used as disposable items or can be regenerated for further use. Thus, the method for the removal of toxins of biological and chemical synthetic origin, their metabolites and degradation products of blood, blood substitutes or solutions for the introduction into the human and/or animal blood circulation can comprise an extra step c):
c) regeneration of the device.

The devices are also used for a method of blood enrichment, blood substitutes or solutions for the introduction into the human and/or animal blood circulation with oxygen and removal of carbon dioxide from blood, blood substitutes or solutions for the introduction into the human and/or animal blood circulation, which comprises the following steps:
a) providing a device for enrichment of blood, blood substitutes or solutions for the introduction into the human and/or animal blood circulation with oxygen and removal of carbon dioxide from blood, blood substitutes or solutions for the introduction into the human and/or animal blood circulation;
b) passage of blood, blood substitutes or solutions for the introduction into the human and/or animal blood circulation, and possibly
c) regeneration of the device.

The devices are preferably used for the simultaneous removal of toxins of biological and chemical synthetic origin, their metabolites and degradation products and carbon dioxide from the blood, blood substitutes or solutions for the introduction into the human and/or animal blood circulation and for enriching the blood, blood substitutes or solutions for the introduction into the human and/or animal blood circulation with oxygen, which comprises the following steps.
a) providing a device for the simultaneous removal of toxins of biological and chemical synthetic origin, their metabolites and degradation products and carbon dioxide from the blood, blood substitutes or solutions for the introduction into the human and/or animal blood circulation and for enriching the blood, blood substitutes or solutions for the introduction into the human and/or animal blood circulation with oxygen;
b) passage of blood, blood substitutes or solutions for the introduction into the human and/or animal blood circulation, and possibly
c) regeneration of the device.
   Endotoxin removal is preferred.

The method includes an extracorporeal procedure. First, one of the devices is wetted with an aqueous solution, if necessary, the wetting agent is washed from the device, filled with a solution tolerable for the patient and then connected via tubes to the bloodstream of the patient. The blood is taken from the patient continuously or discontinuously (single needle application), the toxins of biological and chemical synthetic origin, their metabolites and degradation products from the blood are bound in the device and simultaneously the blood is enriched with oxygen. The treated blood is returned again to the patient continuously or discontinuously.

### EXAMPLES

### Example 1

### Immobilization of Albumin on the Outer Hollow Fiber Surface of a Device with Column I

### 1) Amination of the Outer Hollow Fiber Surface

To prevent that the lumen and the pores of the hollow fiber are also aminated, the column I is filled with a water-immiscible solvent, in this case dodecanol, and then the solvent is drained over the inlets and outlets, which relate to the external surface, and then is gently washed with isotonic saline solution and thereafter with water. The lumen and the pores of the hollow fiber remain filled with the dodecanol, so that it is ensured that only the outer surface of the hollow fiber is aminated in the following.

The cellulose hollow fibers in column I are flushed with a solution of 10% polyethyleneimin solution for 60 minutes at room temperature at a rate of 1 ml/s, such that the solution is passed through the inlet and outlet of the column I, so that only the outer surface of hollow fibers is wetted. Therefore, a ratio of the weights of the hollow fibers to the polyethylenimine solution of 1:2 (w:w) is set. This is followed by washing with isotonic saline, and water till neutrality.

### 2) Immobilization of Albumin

The activation of the carboxyl groups of albumin is conducted with CME-CDI (N-cyclohexyl-N′-(2-morpholinoethyl)-carbodiimide-methyl-p-toluene sulfate). For this purpose a reaction solution of albumin and CME-CDI with a weight ratio of 1:1 (w/w) at 4° C. in 0.1 M MES-buffer (2-(N-morpholino)ethanesulfonic acid) was prepared at pH 4.75 and stirred for half an hour.

The reaction solution is passed for 4 hours at room temperature over the outer surface of the aminated hollow fibers. This is followed by washing with PBS buffer and water to neutrality.

Dodecanol, which is located in the pores and the lumen is removed by air stream and the column I is dried overnight at room temperature.

### Example 2

### Immobilization of Polyamino Acids or Peptides on Polysulfone

Hollow fibers or particles of polysulfone are provided with amino groups as described in J Polym Sci Part A: Polym Chem 41: 1316-1329, 2003, by reaction with n-butyllithium, subsequently with benzonitrile and reduction with cyanoborohydride in acidic medium to benzylamine. The subsequent immobilization of polylysine is achieved, as described in Example 1, by activation of the C-terminal amino acid of polylysine with the carbodiimide CME-CDI and subsequent reaction of the functional groups to the peptide bond.

In the same way, antimicrobial peptides (AMP) and HDL or cholesterol were bound to hollow fibers or particles of polysulfone.

### Example 3

### Immobilization of Heparin on Particles

100 g carrier material in the form of particles of polymethacrylate are incubated with 300 ml of a 25% (w/v) ammonia solution for 3 h at room temperature on a rotary evaporator (use of a stirrer destroys the particles) with slow rotation movements. Then the reaction solution was filtered from the particles and the aminated particles were washed with distilled water to neutrality.

1.5 g of heparin is dissolved completely in a solution of 220 ml of 0.1 M MES-buffer solution and 7.5 g CME-CDI at 4° C. for 30 min at 4° C. This solution is added to the aminated particles and rotated overnight at 4° C.

After this time, the non-covalently bound heparin is flushed with a 4 M aqueous NaCl solution from the modified particles and the modified particles are rinsed thereafter for 30 min with water.

### Example 4

### Filling the Pores of the Particles

Filling the pores of particles with dodecanol prevents the immobilization of substances on the pore surface.

Therefore, the particles are filled into a suitable round bottom flask and dodecanol is added in an amount that the particles are completely covered with dodecanol. After 10 minutes the dodecanol is filtered off. The pores remain filled with dodecanol.

### Example 5

### Immobilization of Heparin on the Outer and Inner Hollow Fiber Surface

First, the pores of the hollow fiber (polyethersulfone) are filled with dodecanol by filling column I completely, i.e. both chambers, and emptying after about 10 minutes. The pores remain filled with dodecanol. The module is then cooled to 4° C.

Initially an amination of the hollow fiber surfaces corresponding to Example 1 is conducted. Afterwards 7.5 mg of CME-CDI is dissolved in 220 ml of 0.1 M MES-buffer pH 4.75 at 4° C., the resulting solution is pumped for 30 min at 4° C. through the column I and is then removed. After rinsing with 250 ml of cold MES-buffer, immobilization solution from 1.5 g of heparin in 275 MES-buffer (pH 4.75) is pumped through the column I overnight at the same temperature.

On the next day, the non-covalently bound heparin is flushed away with 4M NaCl_{aq} and the column I is rinsed with distilled water for 30 minutes. The removal of dodecanol from the pores is achieved with 40° C. warm isopropanol. The heparin coated hollow fibers are rinsed again with water, 4 M NaCl_{aq} and again with water and then left to dry.

### Example 6

### Immobilization of Toxic Shock Syndrome Toxin 1-Binding Peptides (TSST 1-Binding Peptides) on Hollow Fibers

A device with column I having pore sizes of the hollow fibers of 0.65 μm, an inner diameter of the hollow fibers of 0.5 mm and a membrane area of 0.14 m² was flushed in circles with a solution of 94 mg FeSO₄×7 H₂O and 84 mg Na₂S₂O₅ in 200 ml of water. After 15 minutes, first 3.4 ml of methacrylic acid and 2 minutes later 3.4 ml of hydrogen peroxide added (30%) were added into the storage vessel of the solution. Then the solution is pumped 2 hours in circles. Thereafter, the device with column I is flushed with running water for 4 hours to remove the remaining reagents both on the inside and outside of the hollow fibers. The device with column I is completely drained afterwards.

In 220 ml of a 0.1 m MES (2-(N-morpholino) ethanesulfonic acid) buffer solution (pH 4.75), 7.5 g CME-CDI (N-cyclohexyl-N′-(2-morpholinoethyl) carbodiimide methyl-p-toluenesulfonate) at 4° C. is completely dissolved. This solution is pumped at 4° C. for 30 min in circles through column I. Column I is then drained completely and rinsed as quickly as possible with 250 ml of cold 4° C. 0.1 M MES-buffer (pH 4.75). After removing the washing solution, a solution of 1 g of TSST-1 binding peptide (toxic shock syndrome toxin-1-binding peptide, Custom synthesis ordered at Bachem, sequence: GADRSYLSFIHLYPELAGA) in 200 ml of 0.1 M MES-buffer at 4° C. is pumped in circles for 18 hours in the device with column I. Then column I is drained completely and rinsed with water, 4 M sodium chloride solution and again with water and completely dried in vacuum.

The dried device with column I is filled completely with dodecanol and completely emptied after 10 minutes an column I is cooled to 4° C.

### Example 7

### Removal of TSST 1-Binding Peptides from the Outer and Luminal Hollow Fibers Surface

The cooled device with column I, prepared as in Example 6 was filled with 4° C. cold 6 M hydrochloric acid in and around the hollow fibers and stored for 15 hours at 4° C. Column I is then emptied and the constituents of TSST-1 binding peptide are caught in 6 M hydrochloric acid. After that column I was rinsed to pH neutrality with 4° C. cold water and thereafter rinsed with 40° C. warm isopropanol. Finally, it was rinsed again with water, with 4 M sodium chloride solution and again with water and left to dry.

### Example 8

### Coating of the Inner and Outer Surface of Polyetherimide Hollow Fibers with Polyacrylic Acid

### 1. Amination of the Polyetherimide Hollow Fiber Surface

For amination of the surfaces of hollow fibers, both chambers of column I are filled over the inlets slowly, air bubble free with a 4% aqueous solution (degassed distilled water) diethylenamine solution and heated for 30 minutes at 90° C. Then the aminated column I is washed with lots of warm distilled water followed by cold distilled water until pH neutrality and dried.

2. Coating with Polyacrylic Acid

The activation of the carbonyl group of polyacrylic acid is carried out with EDC. For this purpose, 25 g of a 10% polyacrylic acid solution (w/w) are dissolved in 175 g of an isotonic NaCl solution adjusted to pH 4.75. Then the polyacrylic acid solution is mixed with a solution of 2.50 g of N-(3-dimethylaminopropyl)-N-ethylcarbodiimide hydrochloride (EDC) in 100 ml of isotonic NaCl solution and stirred for 45 min at room temperature.

The aminated hollow fibers are added to the activated polyacrylic acid solution and incubated for at least 4 hours at room temperature with the hollow fibers.

### Example 9

### Coating of Hollow Fibers with Endotoxin-Neutralizing-Protein (ENP)

Herefore, ENP is immobilized on the outer surface of polypropylene hollow fibers in column I.

For this purpose, 200 ml of a mixture of ethanol/water 1/1 (v/v) was pumped through the first chamber in column I in circles for 30 minutes at 40° C. Then 4 ml of 3-(triethoxysilyl)-propylamine was added and pumped for further 15 hours at 40° C. in circles. After that washing continued with 200 ml of ethanol/water and 200 ml of water for each 2 hours. 220 mg of the ENP was dissolved at 4° C. in 30 ml of 0.1 M MES-buffer pH 4.75 and mixed with 30 mg of CME-CDI. This solution was pumped for 15 hours at 4° C. in circles through the first chamber into column I. Washing was performed with water, 4 M NaCl solution and water for each 2 hours and dried.

In the same manner as described in this Example 9, polyester hollow fibers and silicone hollow fibers in a column I have been successfully coated with ENP.

### Example 10

### Analysis of the Modified Surfaces of Particles and Hollow Fibers

To determine the levels of substance immobilized on the coated material (particles or hollow fibers), the surface-modified polymers were incubated with 3M hydrochloric acid at 100° C. for 16 h. After removal of hydrochloric acid, the hydrolyzate was separated by anion exchange chromatography.

The signal from a selected component of the formerly immobilized substance is integrated and compared with the signal area of the hydrolyzate of a standard specimen with a defined concentration of the selected substance. The content of immobilized substance onto the samples is calculated from the ratio of the signal area of polymer hydrolysates to standard hydrolysates.

### Example 11

### Surface Modification of Polypropylene Hollow Fibers

The outer surface of polypropylene hollow fibers is coated with albumin. First, the fiber material and the inner space of a column are cleansed with ethanol.

The inner space of the hollow fiber is filled with dodecanol. The covalent binding of albumin on the outer surface of the polypropylene hollow fibers takes place by a modified two-step standard method.

In the first step, an oligomethacrylic acid spacer is covalently attached to polypropylene. In the subsequent step, the albumin coating is bonded to the carboxyl group of the oligomethacrylic acid spacer. 100 mg of albumin were used for the coating of the hollow fibers.

### Example 12

### Surface Modification of Column I

The surface of the first chamber of column I is coated with albumin. As in Example 11, the first chamber of column I is cleansed with ethanol. Then the first chamber of column I is connected by its blood inlet/outlet connections to a peristaltic pump. Initially, 500 ml of a solution of oligomethacrylic acid spacers are pumped through the first chamber of column I in the circuit. Subsequently, 500 ml of albumin solution in the circuit is pumped through the first chamber. Subsequently, the first chamber is rinsed thoroughly with deionized water. 220 mg of albumin is used.

The albumin content of coated polypropylene hollow fibers of Example 11 and samples taken from the coated column I of Example 12 were analyzed as described in Example 10. The results are shown in Table 1.

TABLE 1
Table 1: Comparison of albumin-allocation on polypropylene fibers and
the surface of first chamber of a column I.

| | Polypropylene fibers | Column I |
|---|---|---|
| Polymer | 72 cm² | 5605 cm² |
| Volume of the albumin solution | 200 ml | 500 ml |
| Amount of albumin | 100 mg | 220 mg |
| Allocation of albumin | 32 pmol/cm² | 3.6 pmol/cm² |

The measured content on the surface of the fibers (32 pmol/cm²) is high enough to coat an area of a hypothetical smooth surface which would be twelve times as large.

Thus, it can be stated, that the albumin coating completely covers the surface of the first chamber of a column I.

### Example 13

### Determination of Platelet Loss at Albumin Modified Polypropylene Surfaces

Each 4 ml polypropylene fiber material (PP) and 4 ml albumin surface-modified PP fiber material (prepared as described in Example 1) are dropped each in one 5-ml infusion drop chamber.

The inlets of the drop chambers are connected by a plastic three-way valve. The entire system is flushed with 200 ml of physiological saline (NaCl). The lower arm vein of a blood donor is punctured with a butterfly needle. A blood sample is taken for determination of platelet concentration. Then, the outlet of the butterfly needle is connected by another three-way valve with the free connection of the three-way valve of the NaCl-filled drop chamber. The blood runs freely through the two drop chambers. Through the open port of the three-way valve heparin is added for anticoagulation. The dosis of heparin should be as low as possible and must be determined individually for each experiment. The first outflowing NaCl solution is discarded. The blood flows with at least 3 ml/min and about 50 ml of blood are collected in two holding tanks under the drop chambers. Blood platelet content is also determined.

Determined are
1. Platelet recovery in the drop chamber with the albumin-coated test material
2. Platelet recovery in the drip chamber with the unmodified surface PP fiber material.
3. In an extra measurement platelet recovery is determined in a drop chamber without any filling (reference value).

The platelet recovery for non-modified material is about 52%, for albumin-coated material about 56% and for the empty drop chamber about 84%.

### Example 14

### Surface Modification of Column I

A column I with a polymethyl pentene hollow fiber bundle is connected with its blood inlet/outlet connections to a peristaltic pump. 500 ml of each the polyethylenimine and albumin/CME-CDI-solution (see Example 1) are recirculated through column I, followed by rinsing thoroughly with deionized water. 220 mg of albumin were used.

The determination of the albumin content is performed according to the procedure in Example 10.

### Example 15

### In Vitro Analysis of Endotoxin Adsorption Using a Coated Column I

### Experimental Conditions:

Perfusate: citrated bovine plasma with Endotoxin (150 I.U., LPS from E. coli 055: B5, Sigma-Aldrich)
   pH=7.5; OFSP (surface tension): 53.5±0.8 mN/m
Perfusion rate 10 ml/min
Gas rate 10 ml/min
Gas temperature 22° C.
Perfusion temperature approximately 37° C.
Perfusion time 2 hours

### Preparatory Measures:

The blood compartment of coated column I (first chamber) was purged with CO₂, until no air was in the capillaries and in the blood space. Then the system (first chamber) with connected blood heat exchanger and with oxygen gassing was conditioned with an isotonic saline solution.

### Experiment:

The isotonic saline solution is continuously replaced with the endotoxin containing perfusate. After a perfusion time of 24 hours the endotoxin content in the perfusate or rather after plasma collection is determined by chromogenic Limulus amebocyte lysate assay (LAL assay).

In the same manner as described in this Example 15 adsorption of endotoxins from bovine whole blood, physiological saline and the blood substitute Oxygent® were performed. From all solutions endotoxin could be removed to about 90% with the help of the device.

### Example 16

For devices with column II, the same polymer materials were used as for column I (Examples 1, 2, 3, 5, 6, 8, 11, 12) only with the difference that the pore diameter was bigger (0.01 up to 1.5 μm) to allow the passage of filtrate through the walls of hollow fibers in column II.

The coatings of hollow fibers or particles on column II were made with the same substances and performed in the same manner as described in Examples 1-6, 8, 9 and 12. The same levels of immobilized substance amounts were achieved on the various polymer materials and their coatings.

### Example 17

The adsorption of endotoxins on coated hollow fibers or coated particles was also determined for devices using column II. The adsorption was determined as described in Example 15. Values of about 90% were found for the adsorption of endotoxin from citrated bovine plasma, bovine whole blood, physiological saline solution and blood substitute Oxygent®.

### Example 18

For devices combining column I and column II, the adsorption of endotoxins on coated hollow fibers or coated particles was also determined. Therefore, the two columns were consecutively arranged, so that the endotoxin-containing solution flowed first through column I and then through column II and also vice versa. The adsorption conditions are identical to that mentioned in Example 15. Endotoxin adsorption values from bovine citrated plasma, bovine whole blood, physiological saline solution and from blood substitute Oxygent® were between 95% and 97% by using the device consisting of column I and column II. These data show that the flow direction through the columns I/II has no influence on the degree of adsorption of endotoxin.

### Example 19

Devices combining column I with column II were further analyzed by determining the gas transfer rate for oxygen and carbon dioxide for column I and the effectiveness of hemofiltration for column II.

### Gas Transfer, Column I:

To determine the gas transfer rate sensors for oxygen and carbon dioxide were arranged before the blood inlet and behind the blood outlet of column I. In addition, the gas inlet of column I was provided with an oxygen source. Bovine citrated plasma and bovine whole blood was mixed with well defined small amount of oxygen and a high amount of carbon dioxide. In two consecutive runs changes in oxygen partial pressure and changes in carbon dioxide partial pressure were measured in the oxygenated citrated plasma and in whole blood during circulation through the first chamber of a column I. The two experiments with citrated plasma and whole blood were carried out in column I with uncoated hollow fibers made of polymethyl and for comparison in a column I with polymethyl pentene hollow fibers, which were coated with ENP. The comparison between the columns showed for citrated plasma as well as for whole blood an oxygen transfer into the blood, which was about 20% lower for the column with coated hollow fibers than for the column with uncoated hollow fibers. Similarly, the transfer of carbon dioxide from the blood was about 20% lower for the column with coated hollow fibers than for the column with uncoated hollow fibers.

### Hemofiltration, Column II:

The effectiveness of hemofiltration for column II was determined by measuring the parameters of creatinine, urea and electrolytes sodium and potassium before and after the circulation of bovine citrated plasma or bovine whole blood.

A column II, containing polyethersulfone hollow fibers, coated with albumin was used. The second chamber of this column was provided with an outlet for ultrafiltrate and the circuit of the first chamber, through which the liquid to be filtered flows, was connected to a supply for substitution solution. The concentrations of creatinine, urea and sodium and potassium were adjusted for the liquids to be filtered, bovine citrateplasma and bovine whole blood, as follows:

| creatinine | 3 × 10⁻² | mg/ml |
|---|---|---|
| urea | 2 | mg/ml |
| sodium ions | 250 | mM |
| potassium ions | 10 | mM |

In two consecutive experiments, 500 ml of the fluid to be filtered circulated at a flow rate of 200 ml/min for two hours through the circuit of the first chamber of a column II. After this time the content of the substances listed above was determined. Table 2 shows a compilation of the portions of substances removed out of the liquids to be filtered.

TABLE 2

| | Removed portion |
|---|---|
| Creatinine | 67% |
| Urea | 70% |
| Sodium ions | 61% |
| Potassium ions | 56% |

Thus, the concentrations of all parameters after filtration with column II are within the normal physiological range.

### Example 20

For devices with column I hollow fibers were provided with combined coatings of one hemocompatible substance and one toxin-binding substance.

### 20A. Heparin and ENP on Polymethylpentene Hollow Fibers:

First, the outer surface of the hollow polymethylpentene fibers was aminated as described in Example 1. After that 5 mg of CME-CDI is dissolved in 220 ml of 0.1 M MES-buffer pH 4.75 at 4° C., the resulting solution is pumped for 30 min at 4° C. through the first chamber of column I and then is removed. After rinsing with 250 ml of cold MES-buffer the immobilization solution of 1 g of heparin in 275 MES-buffer (pH 4.75) is pumped overnight at the same temperature through the first chamber of column I. The next day, the non-covalently bound heparin is flushed with 4M NaCl_{aq} from the chamber and then the chamber is washed with water to pH neutrality. As a final step, the ENP coating was performed as described in Example 9.

### 20B. Albumin and Heparin on Polymethacrylate Hollow Fibers:

Amination of the outer surface of the hollow fiber was performed with 300 ml of a solution of 25% (w/v) ammonia solution fed for 3 h at room temperature through the first chamber of column I, which contained the polymethacrylate hollow fibers. Then the first chamber and thus the outer surface of the hollow fibers were washed with water to neutrality. For the first coating step 1 g of heparin was dissolved completely in a solution 220 ml of a 0.1 M MES buffer-solution and 5 g CME-CDI at 4° C. and stirred for 30 min at 4° C. This solution was then pumped overnight at the same temperature through the first chamber of column I. After this time, as described in Example 20A the non-covalently bound heparin was removed from the first chamber and the surface of the hollow fibers. As a final step, the immobilization of albumin on the outer surface of the hollow fibers was performed as described in Example 1.

### Example 21

As indicated in Example 15, the columns I with the combined coatings as shown in examples 20A and 20B were also tested for their binding capacity for endotoxins. At the same time, the transfer of oxygen and carbon dioxide for the columns I from example 20A and 20B were determined as described in Example 19.

Adsorption values of endotoxin from citrated bovine plasma, bovine whole blood, physiological saline solution, and from the blood substitute Oxygent® were about 90% with the use of the device consisting of column I of example 20A or example 20B.

The measured transfer of oxygen and carbon dioxide for the columns I from example 20A and 20B were in the same range as for the column I in Example 19.

### Example 22

The hollow fibers in column II were provided with the same coating, as described in Example 20 for the corresponding column I. The effectiveness of hemofiltration for column II with a combined coating was then determined as described in Example 19.

The effectiveness of hemofiltration for column II with a combined coating was in the same range as the measurements for the column I in Example 19.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. A device for purification of blood, blood substitutes or solutions for the introduction into the human and/or animal blood circulation and for gas exchange in blood, blood substitutes or solutions for the introduction into the human and/or animal blood circulation comprising a column with:
a) an inlet and an outlet for gases or gas mixtures,
b) an inlet and an outlet for blood, blood substitutes or solutions for the introduction into the human and/or animal blood circulation,
c) at least one gas permeable membrane and
d) a carrier, which is coated with substances for adsorptive removal of toxins of biological and chemical synthetic origin, their metabolites and degradation products present in blood, blood substitutes or solutions for the introduction into the human and/or animal blood circulation.

2. The device according to claim 1 comprising another column with:
a) an outlet for filtrate,
b) an inlet and an outlet for blood, blood substitutes or solutions for the introduction into the human and/or animal blood circulation,
c) at least one semipermeable membrane, and
d) a carrier, which is coated with substances for adsorptive removal of toxins of biological and chemical synthetic origin, their metabolites and degradation products present in blood, blood substitutes or solutions for the introduction into the human and/or animal blood circulation.

3. The device according to claim 1, wherein the gas permeable membrane and the coated carrier are combined into one unit.

4. The device according to claim 1, wherein the gas permeable membrane is permeable for oxygen and carbon dioxide.

5. The device according to claim 1, wherein the gas permeable membrane is not permeable for liquids.

6. The device according to claim 1, wherein the gas permeable membrane consists of one or more bundles of hollow fibers.

7. The device according to claim 6, wherein the hollow fibers consist of a material or polymer selected from the group of:
silica, silicones, polyolefins, polytetrafluoroethylene, polyesterurethane, polyetheruretane, polyuerethane, polyethylene terephthalate, polymethylpentane, polymethylpentene, polysaccharides, polypeptides, polyethylenes, polyesters, polystyrenes, polyolefins, polysulfonates, polypropylene, polyethersulfones, polypyrroles, polyvinylpyrrolidone, polysulfones, polylactic acid, polyglycolic acid, polyorthoesters, polyaromatic polyamide, aluminum oxide, glass, sepharose, carbohydrates, copolymers of acrylates or methacrylates and polyamides; polyacrylic ester, polymethacrylic ester, polyacrylamide, Polymethacrylamide, polymethacrylate, polyetherimide, polyacrylonitrile, copolymers of ethylene glycol diacrylate or ethylene glycol dimethacrylate and glycidyl acrylate or glycidyl methacrylate and/or allyl glycidylether, regenerated cellulose, cellulose acetate, hydrophobic polymers with the addition of hydrophilic polymers, derivatives and copolymers of the aforementioned polymers.

8. The device according to claim 6, wherein the hollow fibers of the membrane comprise pores with a diameter in the range of 0.01-5 μm and preferably a diameter of 0.01-1.5 μm.

9. The device according to claim 6, wherein the hollow fibers of the membrane have an outer diameter of about 0.1-1.5 mm, an inner diameter of about 0.1-1 mm and a wall thickness of 5-200 μm, preferably 15-50 μm.

10. The device according to claim 1, wherein the carrier is present in form of particles or in form of hollow fibers.

11. The device according to claim 10, wherein the carrier in form of hollow fibers comprises all properties of the gas permeable membrane.

12. The device according to claim 10, wherein the carrier is present in form of particles and the particles consist of a polymer, selected from the group of:
silica, silicones, polyolefins, polytetrafluoroethylene, polyesterurethane, polyetheruretane, polyuerethane, polyethylene terephthalate, polymethylpentane, polymethylpentene, polysaccharides, polypeptides, polyethylenes, polyesters, polystyrenes, polyolefins, polysulfonates, polypropylene, polyethersulfones, polypyrroles, polyvinylpyrrolidone, polysulfones, polylactic acid, polyglycolic acid, polyorthoesters, polyaromatic polyamide, aluminum oxide, glass, sepharose, carbohydrates, copolymers of acrylates or methacrylates and polyamides; polyacrylic ester, polymethacrylic ester, polyacrylamide, polymethacrylamide, polymethacrylate, polyetherimide, polyacrylonitrile, copolymers of ethylene glycol diacrylate or ethylene glycol dimethacrylate and glycidyl acrylate or glycidyl methacrylate and/or allyl glycidylether, regenerated cellulose, cellulose acetate, hydrophobic polymers with the addition of hydrophilic polymers, derivatives and copolymers of the aforementioned polymers.

13. The device according to claim 10, wherein the carrier is present in form of particles and the particles have a diameter between 50 μm-5 mmm.

14. The device according claim 10, wherein the carrier is present in form of particles and comprises pores with a diameter in the range of 0.01-5 μm and preferably a diameter of 0.01-1.5 μm.

15. The device according to claim 10, wherein the carrier in form of particles has an outer surface and the pores of the carrier in form of particles have an inner surface and the inner surface and the outer surface of the carriers exhibit chemical functional groups.

16. The device according to claim 10, wherein the carrier in form of hollow fibers has an inner surface and an outer surface and the inner surface and the outer surface of the carriers exhibit chemical functional groups.

17. The device according to claim 10, wherein the inner surface and/or the outer surface of the carriers in form of hollow fibers and the inner and/or outer surface of the carriers in form of particles are coated with substances for adsorptive removal of toxins of biological and chemical synthetic origin, their metabolites and degradation products present in blood, blood substitutes or solutions for the introduction into the human and/or animal blood circulation.

18. The device according to claim 1, wherein the substances for adsorptive removal of toxins of biological and chemical synthetic origin, their metabolites and degradation products present in blood, blood substitutes or solutions for the introduction into the human and/or animal blood circulation are bound directly via chemical functional groups or linkers to the surface of the carrier.

19. The device according to claim 1, wherein the substance for adsorptive removal of toxins of biological and chemical synthetic origin, their metabolites and degradation products present in blood, blood substitutes or solutions for the introduction into the human and/or animal blood circulation is selected from the group of polyacrylic acid, derivatives of polyacrylic acid, albumin, metal chelate complexes, cyclodextrins, ion exchangers, polyamino acids, modified polyamino acids, modified and unmodified polyethylenimine, polyallylamine and modified polyallylamine, basic oligopeptides immobilized amidine groups, histidine, polypropylene, polyethylene, polyvinylidene fluoride, polytetrafluoroethylene, alkylaryl groups, monoaminoalkanes, toxic shock syndrome toxin 1-binding peptides, diaminoalkanes, polyaminoalkanes, aromatic nitrogen-containing heterocyclic compounds and their derivatives, antimicrobial peptides, endotoxin-neutralizing protein, synthetic peptides, polylysine, HDL, cholesterol, polymyxin B, polymyxin E, peptides having the formula R-(Lys-Phe-Leu)ₙ-R 1 with R and R₁=H, amino acid residues, membrane-forming lipids, membrane-forming lipoproteins, membrane-forming polysaccharides, membrane forming lipopolysaccharides, glycoproteins, cholesterol esters, triacylglycerols, steroids, phosphoglycerides, sphingolipids, lipoproteins with cyclic residue, lipoproteins without cyclic residue, lipooligosaccharides with protein content, fatty acid residues in length between 1-100 carbon atoms, preferably 1-10 carbon atoms; nitrogen-containing heterocyclic compounds, nitrogen-functionalized aromatic carboxylic acids and/or their derivatives.

20. Use of a device comprising a column with:
a) an inlet and an outlet for gases or gas mixtures,
b) an inlet and an outlet for blood, blood substitutes or solutions for the introduction into the human and/or animal blood circulation,
c) at least one gas permeable membrane, and
d) a carrier, which is coated with substances for adsorptive removal of toxins of biological and chemical synthetic origin, their metabolites and degradation products present in blood, blood substitutes or solutions for the introduction into the human and/or animal blood circulation,
for removal of toxins of biological and chemical synthetic origin, their metabolites and degradation products present in blood, blood substitutes or solutions for the introduction into the human and/or animal blood circulation.

21. Use according to claim 20, wherein the toxins of biological and chemical synthetic origin, their metabolites and degradation products are selected from the group of fibrinogen, toxins according to an infectious disease, toxins in relation with nutrition e.g. fungal toxins, nicotine, ethanol, botulism; toxins from work-related and from criminal acts e.g. lead acetate, B- and C-weapons; toxins in the form of gas, aerosol, liquid and solids such as CO; immune complexes, medicaments, drugs, alcohol, detergents, phosgene, chlorine, hydrogen cyanide, nitrosamines, oxalic acid, benzopyrenes, solanine, nitrates, nitrites, amines, dichlorodisulphide, halogenated hydrocarbons; toxins of bacterial, fungal e.g. mycotoxins as epoxytrichotecene, ochratoxin A, zearalenone; and protozoal origin and their components e.g. exotoxins, endotoxins, fungal spores; and their degradation products, biological warfare toxins such as microcystins, anatoxin, saxitoxin of bacterial origin and their degradation products, insecticides, bactericides, drugs and their metabolites, narcotics, pharmaceuticals and their metabolites and their degradation products, antigens, DNA, RNA, ENA, immunoglobulins, autoimmune antibodies, antibodies, including anti-DNA antibodies, anti-nuclear antibodies, viruses, retroviruses and viral components, such as hepatitis virus particles, lipids, proteins, peptides, proteolipids, glycoproteins and proteoglycans, fibrin, prions, nano weapons, metals, such as Hg, Cd, Pb, Cr, Co, Ni, Zn, Sn, Sb, and ions of these metals, semimetals, such as As; as well as ions of these semi-metals, toxic lipopolysaccharides and endotoxins.

22. Use according to claim 20 for enrichment of blood, blood substitutes or solutions for the introduction into the human and/or animal blood circulation with oxygen.

23. Use according to claim 20 for removal of carbon dioxide out of blood, blood substitutes or solutions for the introduction into the human and/or animal blood circulation.

24. Use according to claim 20 for the simultaneous removal of toxins of biological and chemical synthetic origin, their metabolites and degradation products and carbon dioxide out of blood, blood substitutes or solutions for the introduction into the human and/or animal blood circulation and for the enrichment of blood, blood substitutes or solutions for the introduction into the human and/or animal blood circulation with oxygen.

25. Use according to claim 20 for prophylaxis, alleviation or treatment of diseases that are caused by toxins of biological and chemical synthetic origin, their metabolites and degradation products

26. Use according to claim 20 for prophylaxis, alleviation or treatment of diseases that are due to the presence of lipopolysaccharides or endotoxins as membrane fragments of gram-negative bacteria.

27. Use according to claim 20, wherein the diseases caused by toxins of biological and chemical synthetic origin, their metabolites and degradation products or that are due to the presence of lipopolysaccharides or endotoxins as membrane fragments of gram-negative bacteria, are selected from the group of: Endotoxemia, sepsis, fever, inflammation, organ failure, multiple organ failure, coagulopathy, rhabdomyolysis, necrosis, shock, trauma, bacteremia, diarrhea, leukocytosis, vasodilation, coagulation due to hypotension, circulatory failure, systemic inflammatory response syndrome, adult respiratory distress syndrome.

28. Use according to claim 27, wherein the disease is sepsis.

29. A process for removal of toxins of biological and chemical synthetic origin, their metabolites and degradation products out of blood, blood substitutes or solutions for the introduction into the human and/or animal blood circulation, comprising the steps:
a) providing a device for removal of toxins of biological and chemical synthetic origin, their metabolites and degradation products out of blood, blood substitutes or solutions for the introduction into the human and/or animal blood circulation;
b) passage of blood, blood substitutes or solutions for the introduction into the human and/or animal blood circulation.

30. Process according to claim 29 further comprising the step c):
c) regeneration of the device.
